(19) United States
(12) Patent Application Publication
(10) Pub. No.: US 2022/0144938 A1
(43) Pub. Date: May 12, 2022

(54) IDENTIFICATION AND TARGETING OF TUMOR PROMOTING CARCINOMA ASSOCIATED FIBROBLASTS FOR DIAGNOSIS AND TREATMENT OF CANCER AND OTHER DISEASES

(71) Applicant: Board of Regents, The University of Texas System, Austin, TX (US)

(72) Inventor: Raghu Kalluri, Houston, TX (US)

(21) Appl. No.: 17/299,265

(22) PCT Filed: Dec. 6, 2019
(86) PCT No.: PCT/US2019/065008

§ 371 Date: Jun. 2, 2021

Related U.S. Application Data
(60) Provisional application No. 62/777,101, filed on Dec. 8, 2018.

(51) Int. Cl. C07K 16/24; A61K 45/06; C07K 16/28; A61K 39/395; G01N 33/574; A61K 31/7068; A61P 35/00

(52) U.S. Cl. CPC C07K 16/248; A61K 45/06; C07K 16/2809; A61K 39/3955; A61K 2039/505; A61K 31/7068; A61P 35/00; C07K 2317/76; G01N 33/57438

(57) ABSTRACT

Provided herein are agents, such as antibodies or chimeric antigen receptors, that target TP-CAFs. Methods of treating cancer are provided, comprising administering to a patient in need thereof an effective amount of a TP-CAFs-neutralizing agent. The methods can further include administering an effective amount of chemotherapy or immunotherapy to said patient. The methods can include administering an IL-6 signaling inhibitor in combination with an immune checkpoint blockade therapy.

### REFERENCE TO RELATED APPLICATIONS

The present application claims the priority benefit of U.S. provisional application No. 62/777,101, filed Dec. 8, 2018, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Dec. 6, 2019, is named UTFCP1429WO_ST25.txt and is 2.0 kilobytes in size.

### BACKGROUND

### 1. Field

The present invention relates generally to the field of medicine. More particularly, it concerns methods of treating cancer by targeting tumor-promoting cancer associated fibroblasts and/or by inhibiting IL-6 signaling in combination with immune checkpoint blockage therapy.

### 2. Description of Related Art

Fibroblasts accumulate in tumors with a putative capacity to regulate PDAC progression (LeBleu & Kalluri, 2018; Kalluri, 2016). Collectively, they are referred to as cancer associated fibroblasts (CAFs). CAFs can function to orchestrate a host response to cancer, via cooperation with immune cells and cancer cells, and impact PDAC progression and/or response to treatment. The biology of CAFs in PDAC is evolving, with increased recognition of their role in shaping the tumor immune microenvironment (Kalluri, 2016; Neesse et al., 2015; Ohlund et al., 2014). The αSMA⁺ CAFs function in restraining tumors in genetically engineered mouse models (GEMMs) of PDAC and they polarize tumor infiltrating T cells (Ozdemir et al., 2014).

Recent studies highlighted a distinct subtype of CAFs in PDAC, defined by the expression of FAP, which act as an immune polarizer to suppress PDAC tumors, possibly via CXCL12 (SDF1) (Feig et al., 2013) as well as CCL2 signaling (Yang et al., 2016). Loss of FAP protein delayed PDAC disease progression in mice (Lo et al., 2017); however, targeting FAP⁺ CAFs can also yield cachexic phenotypes, bone toxicity, and anemia (Roberts et al., 2013; Tran et al., 2013). As such, new methods of diagnosing and treating cancer by targeting CAFs are needed.

### SUMMARY

To offer a comprehensive and functional definition of PDAC CAFs, CAFs identity and functions were ascertained using novel GEMMs, multispectral imaging analyses of multiple CAF biomarkers, and single cell RNA sequencing of isolated CAF populations and human and mouse PDAC tumors. CAFs were found to be functionally heterogeneous within the tumor microenvironment and to have opposing functions. Additionally, αSMA⁺ CAFs-derived interleukin-6 (IL-6) was identified as a negative regulator of T cell mediated anti-tumor response during chemotherapy and immune checkpoint blockade.

Fibroblasts are a heterogeneous population comprising tumor restraining fibroblasts/mesenchymal cells and tumor promoting fibroblasts/mesenchymal cells. Several genes/proteins that are specifically associated with tumor promoting fibroblasts are not present in the tumor restraining fibroblasts/mesenchymal cells. As such, therapeutic agents that can identify and target these identified genes/proteins can synergize with chemotherapy, radiation therapy, and immune checkpoint blockade. In addition, TP-CAF-specific CAR-T constructs in autologous T cells or autologous or allogeneic NK cells may be used as an immunotherapy approach. ShRNA, siRNA, and CRISPR-CAS-9 targeting may also be employed. Bispecific antibodies that target TP-CAF via one arm and CD3 via the other arm may lead to immune-targeting of TP-CAF to control cancer progression.

In one embodiment, provided herein are compositions comprising an antibody or an antibody fragment or a chimeric antigen receptor that binds to a protein that is expressed by TP-CAFs and is not expressed by TS-CAFs. The protein may be any one of Apoe; Fth1; Ftl1; Tmsb4x; Rpl41; Rps29; Actb; Rps27; Rps28; Lyz2; Rpl37a; mt-Atp6; mt-Co1; Rps19; Rpl13; Rplp0; Rpl32; Fau; Rpl18a; mt-Co3; Cd74; Rpl35; Rps18; Rpl39; Rpl13a; Rpl37; Tmsb10; Rps23; Rpl35a; Rplp1; Rps15a; Rpl36; Gm8730; Cxcl2; Rps5; Rps27a; Gm10260; Rps16; Rps24; Rpl38; Rps4x; Rplp2; Rps9; Rpl17; Rps11; Rpl10; Rps6; Cebpb; Rps14; Rpl26; Rps8; Rpl34; S100a8; Rpl6; Gm9843; Rps3a1; mt-Nd1; Rps27rt; Rpl14; Rpl27a; Rpl9; Rps7; Rpl23; Rpl19; Rps3; H2-Aa; Tyrobp; Cst3; Tpt1; mt-Co2; Eef1a1; Rpl11; Rpl8; Wfdc17; Pfn1; Rpl3; Rps13; S100a9; Rpl21; Rpl24; Rpl23a; Rps26; C1qa; Rps18-ps3; Fcer1g; Rpl36a; Rps15; Uba52; Gm11808; Gm2000; Rpl15; Rps20; Rpl27; Rpl10a; Rps10; H2-D1; B2m; H2-Ab1; Rpl31; Ccl6; Wdr89; Rpl28; Rps12; Rpl18; Rpl29; Rpsa; Oaz1; Btg1; Rps21; Rps17; C1qc; Ctsd; Psap; Rpl4; Rps25; Ctsb; Cox8a; C1qb; Atox1; H2afz; Ctss; Rpl12; Cfl1; Actg1; Cox4i1; Rpl10-ps3; Rpl7; Gpx1; Cyba; Gm10116; Gm9493; H3f3a; Rpl30; Atp5e; Rpl23a-ps3; Srgn; Cd14; Rpl9-ps6; Rpl13-ps3; Arpc1b; Rpl61; Thbs1; Trf; mt-Nd4; Il1b; Rpl22; Sh3bgrl3; Sepp1; Shfm1; Lgals3; Npc2; Sat1; Pim1; Rps12-ps3; Sub1; Cd52; Gm10076; Gng5; Cstb; Rps26-ps1; Msrb1; Cox6b1; Rpl36a1; Arpc2; Pf4; Lamp1; Naca; Ucp2; Prdx5; Snrpg; Gm10073; Id2; Bcl2a1b; H2-K1; Pabpc1; Fxyd5; Lgmn; Rpl27-ps3; Ndufa2; Ctsz; Cox6a1; Uqcr11; Npm1; Gnb2l1; Eif3f; Ccl8; Ube2d3; Fcgr3; Coal; Gm10263; AA467197; Uqcrq; Gnai2; Ubl5; D8Ertd738e; Ndufa3; Ccl3; Sdcbp; Serinc3; Fcgr2b; Tomm7; Atp6v1g1; Cox7a21; Clta; Grn; Ccrl2; Atp5g2; G0s2; Ptpn18; Smdt1; Bri3; Jchain; Rgs10; Atp6v0e; Rap1b; Rbm39; Laptm5; Rpl5; Atp6v0b; Serp1; Akr1a1; Mcl1; Ccl7; Ccl9; Eef1b2; Marcksl1; Mrp152; Cd53; Atp5k; Pnrc1; Myeov2; Sdc4; Clec4n; Tma7; Cdc42; Rnaset2a; Rac2; 2010107E04Rik; Arpc3; Alox5ap; Vamp8; Zfp36l2; Tspo; Ctsh; Atp6v1f; Coro1a; Ninj1; Ccl4; Rnf149; Tgfbi; Fosl2; mt-Nd2; Ms4a6c; Ndufa6; Pcbp2; Klf13; Eif3h; Ostf1; Hilpda; Fam49b; Trem2; Ctsc; Rgs1; Mafb; Prelid1; Fabp5; Ndufa1; Cox17; Eno1; Gm2a; Hnrnpf; Gdi2; Eif3e; Retnlg; Picalm; Ndufb7; Cox5a; Kdm6b; Acp5; Arhgdib; Plaur; Arg1; Srp9; Tomm20; Timm13; Crem; Lst1; Arpc5; Bola2; Hmgb2; Hexa; Gm10036; Cxcl16; Mtdh; Lcp1; Spi1; Gm6576; Sh3glb1; Ndufb8; Gdpd3; Hn1; Cirbp; Plek; Hspe1; Bcl2a1d; Capza2; Ctsa; Efhd2; Gm42418; Gm8186; Tpd52; Tra2b; Actr3; Sptssa; Brk1; Ppp1ca; Ndufv3; Erdr1; Arpc4; Cdk2ap2; Zfos1; Snx3; Ccl17; Ap2s1; Rac1; Cxcr4; Eif5; and Pitpna.

In some aspects, the antibody fragment is a recombinant scFv (single chain fragment variable) antibody, Fab fragment, F(ab′)₂ fragment, or Fv fragment. In some aspects, the antibody is a chimeric antibody or is a bispecific antibody. In some aspects, the chimeric antibody is a humanized antibody. In some aspects, the bispecific antibody binds to both (1) a protein that is expressed by TP-CAFs and is not expressed by TS-CAFs and (2) CD3. In some aspects, the antibody or antibody fragment is conjugated to a cytotoxic agent. In some aspects, the antibody or antibody fragment is conjugated to a diagnostic agent. In one embodiment, provided herein are hybridomas or engineered cells encoding an antibody or antibody fragment of any one of the present embodiments. In one embodiment, provided herein are pharmaceutical formulations comprising one or more antibody or antibody fragment or chimeric antigen receptor of any one of the present embodiments.

In one embodiment, provided herein are methods of treating a patient in need thereof, the method comprising administering an effective amount of an antibody or an antibody fragment or a chimeric antigen receptor that binds to a protein that is expressed by TP-CAFs and is not expressed by TS-CAFs. The protein may be any one of Apoe; Fth1; Ftl1; Tmsb4x; Rpl41; Rps29; Actb; Rps27; Rps28; Lyz2; Rpl37a; mt-Atp6; mt-Co1; Rps19; Rpl13; Rplp0; Rpl32; Fau; Rpl18a; mt-Co3; Cd74; Rpl35; Rps18; Rpl39; Rpl13a; Rpl37; Tmsb10; Rps23; Rpl35a; Rplp1; Rps15a; Rpl36; Gm8730; Cxcl2; Rps5; Rps27a; Gm10260; Rps16; Rps24; Rpl38; Rps4x; Rplp2; Rps9; Rpl17; Rps11; Rpl10; Rps6; Cebpb; Rps14; Rpl26; Rps8; Rpl34; S100a8; Rpl6; Gm9843; Rps3a1; mt-Nd1; Rps27rt; Rpl14; Rpl27a; Rpl9; Rps7; Rpl23; Rpl19; Rps3; H2-Aa; Tyrobp; Cst3; Tpt1; mt-Co2; Eef1a1; Rpl11; Rpl8; Wfdc17; Pfn1; Rpl3; Rps13; S100a9; Rpl21; Rpl24; Rpl23a; Rps26; C1qa; Rps18-ps3; Fcer1g; Rpl36a; Rps15; Uba52; Gm11808; Gm2000; Rpl15; Rps20; Rpl27; Rpl10a; Rps10; H2-D1; B2m; H2-Ab1; Rpl31; Ccl6; Wdr89; Rpl28; Rps12; Rpl18; Rpl29; Rpsa; Oaz1; Btg1; Rps21; Rps17; C1qc; Ctsd; Psap; Rpl4; Rps25; Ctsb; Cox8a; C1qb; Atox1; H2afz; Ctss; Rpl12; Cfl1; Actg1; Cox4i1; Rpl10-ps3; Rpl7; Gpx1; Cyba; Gm10116; Gm9493; H3f3a; Rpl30; Atp5e; Rpl23a-ps3; Srgn; Cd14; Rpl9-ps6; Rpl13-ps3; Arpc1b; Rpl61; Thbs1; Trf; mt-Nd4; Il1b; Rpl22; Sh3bgrl3; Sepp1; Shfm1; Lgals3; Npc2; Sat1; Pim1; Rps12-ps3; Sub1; Cd52; Gm10076; Gng5; Cstb; Rps26-ps1; Msrb1; Cox6b1; Rpl36a1; Arpc2; Pf4; Lamp1; Naca; Ucp2; Prdx5; Snrpg; Gm10073; Id2; Bcl2a1b; H2-K1; Pabpc1; Fxyd5; Lgmn; Rpl27-ps3; Ndufa2; Ctsz; Cox6a1; Uqcr11; Npm1; Gnb2l1; Eif3f; Ccl8; Ube2d3; Fcgr3; Coal; Gm10263; AA467197; Uqcrq; Gnai2; Ubl5; D8Ertd738e; Ndufa3; Ccl3; Sdcbp; Serinc3; Fcgr2b; Tomm7; Atp6v1g1; Cox7a21; Clta; Gm; Ccrl2; Atp5g2; G0s2; Ptpn18; Smdt1; Bri3; Jchain; Rgs10; Atp6v0e; Rap1b; Rbm39; Laptm5; Rpl5; Atp6v0b; Serp1; Akr1a1; Mcl1; Ccl7; Ccl9; Eef1b2; Marcksl1; Mrp152; Cd53; Atp5k; Pnrc1; Myeov2; Sdc4; Clec4n; Tma7; Cdc42; Rnaset2a; Rac2; 2010107E04Rik; Arpc3; Alox5ap; Vamp8; Zfp36l2; Tspo; Ctsh; Atp6v1f; Coro1a; Ninj1; Ccl4; Rnf149; Tgfbi; Fosl2; mt-Nd2; Ms4a6c; Ndufa6; Pcbp2; Klf13; Eif3h; Ostf1; Hilpda; Fam49b; Trem2; Ctsc; Rgs1; Mafb; Prelid1; Fabp5; Ndufa1; Cox17; Eno1; Gm2a; Hnrnpf; Gdi2; Eif3e; Retnlg; Picalm; Ndufb7; Cox5a; Kdm6b; Acp5; Arhgdib; Plaur; Arg1; Srp9; Tomm20; Timm13; Crem; Lst1; Arpc5; Bola2; Hmgb2; Hexa; Gm10036; Cxcl16; Mtdh; Lcp1; Spi1; Gm6576; Sh3glb1; Ndufb8; Gdpd3; Hn1; Cirbp; Plek; Hspe1; Bcl2a1d; Capza2; Ctsa; Efhd2; Gm42418; Gm8186; Tpd52; Tra2b; Actr3; Sptssa; Brk1; Ppp1ca; Ndufv3; Erdr1; Arpc4; Cdk2ap2; Zfos1; Snx3; Ccl17; Ap2s1; Rac1; Cxcr4; Eif5; and Pitpna.

In some aspects, the antibody or antibody fragment or chimeric antigen receptor that binds to a protein that is expressed by TP-CAFs and is not expressed by TS-CAFs is the antibody or antibody fragment or chimeric antigen receptor of any one of the present embodiments. In some aspects, the patient has a cancer. In some aspects, the cancer has been determined to comprise FAP⁺ CAFs. In some aspects, the cancer is a pancreatic cancer. In some aspects, the methods are methods of inhibiting pancreatic cancer metastasis. In some aspects, the methods are methods of inhibiting pancreatic cancer growth. In some aspects, the methods further comprise administering at least a second anti-cancer therapy. In some aspects, the second anti-cancer therapy is a chemotherapy, immunotherapy, radiotherapy, gene therapy, surgery, hormonal therapy, anti-angiogenic therapy or cytokine therapy.

In one embodiment, provided herein are chimeric antigen receptor (CAR) polypeptides comprising, from N- to C-terminus, an antigen binding domain; a hinge domain; a transmembrane domain and an intracellular signaling domain, wherein the CAR polypeptide binds to a protein that is expressed by TP-CAFs and is not expressed by TS-CAFs. The protein may be any one of Apoe; Fth1; Ftl1; Tmsb4x; Rpl41; Rps29; Actb; Rps27; Rps28; Lyz2; Rpl37a; mt-Atp6; mt-Co1; Rps19; Rpl13; Rplp0; Rpl32; Fau; Rpl18a; mt-Co3; Cd74; Rpl35; Rps18; Rpl39; Rpl13a; Rpl37; Tmsb10; Rps23; Rpl35a; Rplp1; Rps15a; Rpl36; Gm8730; Cxcl2; Rps5; Rps27a; Gm10260; Rps16; Rps24; Rpl38; Rps4x; Rplp2; Rps9; Rpl17; Rps11; Rpl10; Rps6; Cebpb; Rps14; Rpl26; Rps8; Rpl34; S100a8; Rpl6; Gm9843; Rps3a1; mt-Nd1; Rps27rt; Rpl14; Rpl27a; Rpl9; Rps7; Rpl23; Rpl19; Rps3; H2-Aa; Tyrobp; Cst3; Tpt1; mt-Co2; Eef1a1; Rpl11; Rpl8; Wfdc17; Pfn1; Rpl3; Rps13; S100a9; Rpl21; Rpl24; Rpl23a; Rps26; C1qa; Rps18-ps3; Fcer1g; Rpl36a; Rps15; Uba52; Gm11808; Gm2000; Rpl15; Rps20; Rpl27; Rpl10a; Rps10; H2-D1; B2m; H2-Ab1; Rpl31; Ccl6; Wdr89; Rpl28; Rps12; Rpl18; Rpl29; Rpsa; Oaz1; Btg1; Rps21; Rps17; C1qc; Ctsd; Psap; Rpl4; Rps25; Ctsb; Cox8a; C1qb; Atox1; H2afz; Ctss; Rpl12; Cfl1; Actg1; Cox4i1; Rpl10-ps3; Rpl7; Gpx1; Cyba; Gm10116; Gm9493; H3f3a; Rpl30; Atp5e; Rpl23a-ps3; Srgn; Cd14; Rpl9-ps6; Rpl13-ps3; Arpc1b; Rpl61; Thbs1; Trf; mt-Nd4; Il1b; Rpl22; Sh3bgrl3; Sepp1; Shfm1; Lgals3; Npc2; Sat1; Pim1; Rps12-ps3; Sub1; Cd52; Gm10076; Gng5; Cstb; Rps26-ps1; Msrb1; Cox6b1; Rpl36a1; Arpc2; Pf4; Lamp1; Naca; Ucp2; Prdx5; Snrpg; Gm10073; Id2; Bcl2a1b; H2-K1; Pabpc1; Fxyd5; Lgmn; Rpl27-ps3; Ndufa2; Ctsz; Cox6a1; Uqcr11; Npm1; Gnb2l1; Eif3f; Ccl8; Ube2d3; Fcgr3; Cotl1; Gm10263; AA467197; Uqcrq; Gnai2; Ubl5; D8Ertd738e; Ndufa3; Ccl3; Sdcbp; Serinc3; Fcgr2b; Tomm7; Atp6v1g1; Cox7a21; Clta; Gm; Ccrl2; Atp5g2; G0s2; Ptpn18; Smdt1; Bri3; Jchain; Rgs10; Atp6v0e; Rap1b; Rbm39; Laptm5; Rpl5; Atp6v0b; Serp1; Akr1a1; Mcl1; Ccl7; Ccl9; Eef1b2; Marcksl1; Mrp152; Cd53; Atp5k; Pnrc1; Myeov2; Sdc4; Clec4n; Tma7; Cdc42; Rnaset2a; Rac2; 2010107E04Rik; Arpc3; Alox5ap; Vamp8; Zfp36l2; Tspo; Ctsh; Atp6v1f; Coro1a; Ninj1; Ccl4; Rnf149; Tgfbi; Fosl2; mt-Nd2; Ms4a6c; Ndufa6; Pcbp2; Klf13; Eif3h; Ostf1; Hilpda; Fam49b; Trem2; Ctsc; Rgs1; Math; Prelid1; Fabp5; Ndufa1; Cox17; Eno1; Gm2a; Hnrnpf; Gdi2; Eif3e; Retnlg; Picalm; Ndufb7; Cox5a; Kdm6b; Acp5; Arhgdib; Plaur; Arg1; Srp9; Tomm20; Timm13; Crem; Lst1; Arpc5; Bola2; Hmgb2; Hexa; Gm10036; Cxcl16; Mtdh; Lcp1; Spi1; Gm6576; Sh3g1b1; Ndufb8; Gdpd3; Hn1; Cirbp; Plek; Hspe1; Bcl2a1d; Capza2; Ctsa; Efhd2; Gm42418; Gm8186; Tpd52; Tra2b; Actr3; Sptssa; Brk1; Ppp1ca; Ndufv3; Erdr1; Arpc4; Cdk2ap2; Zfos1; Snx3; Ccl17; Ap2s1; Rac1; Cxcr4; Eif5; and Pitpna.

In some aspects, the antigen binding domain comprises HCDR sequences from a first antibody that binds to a protein that is expressed by TP-CAFs and is not expressed by TS-CAFs and LCDR sequences from a second antibody that binds to a protein that is expressed by TP-CAFs and is not expressed by TS-CAFs. In some aspects, the antigen binding domain comprises HCDR sequences and LCDR sequence from an antibody that binds to a protein that is expressed by TP-CAFs and is not expressed by TS-CAFs. In some aspects, the hinge domain is a CD8a hinge domain or an IgG4 hinge domain. In some aspects, the transmembrane domain is a CD8a transmembrane domain or a CD28 transmembrane domain. In some aspects, the intracellular signaling domain comprises a CD3z intracellular signaling domain.

In one embodiment, provided herein are nucleic acid molecules encoding a CAR polypeptide of any one of any one of the present embodiments. In some aspects, the sequence encoding the CAR polypeptide is operatively linked to expression control sequences. In one embodiment, provided herein are isolated immune effector cells comprising a CAR polypeptide according to any one of the present embodiments or a nucleic acid of any one of the present embodiments. In some aspects, the nucleic acid is integrated into the genome of the cell. In some aspects, the cell is a T cell. In some aspects, the cell is an NK cell. In some aspects, the cell is a human cell. In one embodiment, provided herein are pharmaceutical compositions comprising a population of cells in accordance with any one of the present embodiments in a pharmaceutically acceptable carrier.

In one embodiment, provided herein are methods of treating a subject comprising administering an anti-tumor effective amount of chimeric antigen receptor (CAR) T cells that expresses a CAR polypeptide in accordance with any one of the present embodiments. In some aspects, the CAR T cells are allogeneic cells. In some aspects, the CAR T cells are autologous cells. In some aspects, the CAR T cells are HLA matched to the subject. In some aspects, the subject has a cancer. In some aspects, the cancer is a pancreatic cancer.

In one embodiment, provided herein are methods of treating a subject comprising administering an anti-tumor effective amount of chimeric antigen receptor (CAR) NK cells that expresses a CAR polypeptide in accordance with any one of the present embodiments. In some aspects, the CAR NK cells are allogeneic cells. In some aspects, the CAR NK cells are autologous cells. In some aspects, the CAR NK cells are HLA matched to the subject. In some aspects, the subject has a cancer. In some aspects, the cancer is a pancreatic cancer.

In one embodiment, provided herein are methods of diagnosing a patient as having a disease, the method comprising contacting a cancer tissue obtained from the subject with an antibody or antibody fragment of any one of the present embodiments and detecting the binding of the antibody or antibody fragment to the tissue, wherein if the antibody or antibody fragment binds to the tissue, then the patient is diagnosed as having a cancer.

In one embodiment, provided herein are methods of treating a subject having a disease, the method comprising administering an anti-tumor effective amount of a composition that comprises an agent that suppresses IL-6 signaling, gemcitabine, and an immune checkpoint blockade therapy. In some aspects, the disease is a cancer. In some aspects, the cancer has previously failed to respond to immune checkpoint blockade therapy. In some aspects, the methods further comprise administering an effective amount of an antibody or an antibody fragment or a chimeric antigen receptor that binds to a protein that is expressed by TP-CAFs and is not expressed by TS-CAFs. In some aspects, the cancer is a pancreatic cancer. In some aspects, the methods are methods of inhibiting pancreatic cancer metastasis. In some aspects, the methods are methods of inhibiting pancreatic cancer growth. In some aspects, the methods further comprise administering at least a second anti-cancer therapy. In some aspects, the second anti-cancer therapy is a chemotherapy, immunotherapy, radiotherapy, gene therapy, surgery, hormonal therapy, anti-angiogenic therapy or cytokine therapy.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. CAFs heterogeneity in PDAC tumors. FIG. 1A. Representative stromal cell composition in PKT tumors (n=11 mice). These data are also shown in FIG. 4E. FIG. 1B. Quantification of co-localization by immunofluorescent labeling of αSMA and FAP in human PDAC (n=2 patients, standard deviation represents variation of staining distribution across visual fields). FIG. 1C-D. Characterization of αSMA-RFP⁺ cells, YFP⁺ cancer cells, and FAP-APC immunolabeled cells in the tumor of PKT; LSL-YFP; αSMA-RFP GEM. n=1 mouse, sorted cell populations were subsequently used in scRNA seq analyses (FIGS. 2A-C). GEM: genetically engineered mice, Neg: negative, s+: single positive, Vim: vimentin, scRNA seq: single cell RNA sequencing. See Table 1 for mouse GEM nomenclature.

FIGS. 2A-B. αSMA⁺ and FAP⁺ CAFs define distinct fibroblasts subpopulations with immune-like transcriptomic profiles. FIG. 2A. scRNA seq analyses of αSMA⁺ and FAP cells enriched by flow cytometry (FIG. 1D) from PKT tumors represented as t-SNE plots, with clusters (1, 2, 3, 4 & 6, left panel) from αSMA⁺ enriched cells and clusters (3, 5, 7, left panel) from FAP⁺ enriched cells. Cluster 3 comprises a cluster of cells with shared transcriptomic identity between αSMA⁺ and FAP⁺ enriched cells. Functional clusters (1-9, right panel) were also defined and group definition listed. FIG. 2B. Specific αSMA⁺ and FAP⁺ clusters were comparatively profiled and gene networks identified based on enriched transcripts in each cluster. scRNA seq: single cell RNA sequencing, RBC: red blood cells, ECM: extracellular matrix.

FIG. 3. Cellular heterogeneity captured by scRNA sequencing of human of PDAC tumors. scRNA seq analyses of unfractionated human PDAC tumors represented as t-SNE plots, two patients were evaluated (PDAC 1 and PDAC 2). For PDAC 2, two distinct flow cytometry cell sortings were carried out (PDAC 2A, PDAC 2B; technical replicates), and subsequently merged into PDAC 2 for subsequent analyses. scRNA seq: single cell RNA sequencing.

FIGS. 4A-E. Distinct outcomes on PDAC progression by selective depletion of αSMA⁺ vs FAP⁺ CAFs. FIG. 4A. Bar graphs depicting the histological features of the indicated groups following H&E staining of pancreas tumor sections of PKT GEMs with and without αSMA⁺ or FAP⁺ cell depletion. Depletion was enabled by GCV administration in PKT mice harboring the αSMA-TK and FAP-TK transgene. Controls include PKT mice harboring the transgene and administered with PBS or not injected, as well as PKT mice without the transgene and administered GCV. FAP-TK, n=5 and control, n=8; αSMA-TK, n=11 and control, n=17. The mean+/−standard error of the mean is depicted. Statistical significance was evaluated using the Mann-Whitney test. FIG. 4B. Bar graphs depicting quantification of immunohistochemistry (IHC) for αSMA and FAP in PKT tumors and associated quantification in the indicated groups. FAP-TK, n=6 and control, n=7; αSMA-TK, n=5 and control, n=5 mice. Statistical significance was evaluated using the Mann-Whitney test. FIGS. 4C-D. Overlap in genes (FIG. 4C) and associated pathways (FIG. 4D) commonly down- or up-regulated in tumors of PKT mice with αSMA⁺ vs. FAP⁺ cell depletion. FAP-TK, n=3 and control, n=3; αSMA-TK, n=3 and control, n=3 mice. FIG. 4E. Mesenchymal cell composition in PKT tumors (also shown in FIG. 1A) and PKT tumors depleted of either αSMA⁺ or FAP⁺ cells. FAP-TK, n=7; αSMA-TK, n=5, control, n=4. Control (combined control for both FAP-TK^{control}, n=7, αSMA-TK^{control}, n=4), n=11. 48.3%*: significant difference evaluated by unpaired two-tailed t test. The bar graph depicts quantitation of αSMA stromal depletion, Mann-Whitney test. The mean+/−standard deviation is depicted. GEM: genetically engineered mice, GCV: ganciclovir, IRS: immune reactive score. See Table 1 for mouse GEM nomenclature.

FIGS. 5A-H. IL-6 from αSMA⁺ CAFs confers cancer cell resistance to gemcitabine. FIG. 5A. scRNA seq analyses of αSMA⁺ and FAP⁺ enriched cells from PKT tumors represented as t-SNE plots and indicating the number of cells (in parenthesis) and percentages of them expressing IL-6. The bar graph summarizes the data obtained from the scRNA seq, supporting IL-6 transcripts are predominantly enriched in αSMA⁺ cells. FIG. 5B. qPCR quantitation of IL-6 transcripts in the indicated cells. Cells were obtained from 3 distinct mice. Statistical significance was evaluated using unpaired two-tailed t test. FIG. 5C. Quantification of the tumor histological phenotype of H&E sections of the pancreas tumor in the indicated GEM. Within each column, the sections represent, from top to bottom, necrosis, poor, well, PanIN, and Normal. Two-way ANOVA. FIG. 5D. Survival of the indicated GEM over time. Log rank test. See FIG. 6C. FIG. 5E. qPCR quantitation of IL-6 transcripts in the tumors, n=4 mice per group. From left to right, the bars represent KPPF, KPPF;IL-6^{smaKO}, and KPPF;IL-6^{-/-}. Statistical significance was evaluated using unpaired two-tailed t test. FIG. 5F. Survival of the indicated GEM over time. From left to right at the 50% survival mark on the y-axis, the lines represent KPPF Gem, KPPF Gem IL-6, KPPF;IL-6^{smaKO}, and KPPF;IL-6^{-/-}. Log rank test. See FIG. 6C. FIG. 5G. qPCR quantitation of IL-6 and Acta2 (αSMA) transcripts in the tumors, n=3 mice per group. Statistical significance was evaluated using unpaired two-tailed t test. FIG. 5H. Quantitative analyses of the number of phospho-Stat3⁺ cells per visual field following immunohistochemistry for phosphorylated Stat3 (phospho-Stat3) in the indicated GEM. The bars represent, from left to right, KPPF, KPPF;IL-6^{smaKO}, KPPF;IL-6^{-/-}, KPPF Gem, and KPPF;IL-6^{smaKO} Gem. n=5 mice per group, one-way ANOVA. The mean+/−standard error of the mean is shown. * P<0.05, ** P<0.01, *** P<0.005, **** P<0.001, ns: not significant. scRNAseq: single cell RNA sequencing, GEM: genetically engineered mice, Gem: gemcitabine, qPCR: quantitative PCR, nd: not detected. See Table 1 for mouse GEM nomenclature.

FIGS. 6A-D. The benefit of stromal IL-6 polarization of intratumoral T cells is realized concurrently with gemcitabine. FIG. 6A. Tumor immune infiltrate fractions in the indicated GEM and treatment group. Data are presented as the mean+/−standard error of the mean, unpaired one-tailed t test. FIG. 6B. Survival of mice in the indicated experimental groups. From left to right, at the 25% survival mark on the y-axis, the lines represent KPPF Gem, KPPF Gem CP, KPPF;IL-6^{-/-} Gem, and KPPF;IL-6^{-/-} Gem CP. Log rank test, see FIG. 6C. FIG. 6C. Overview of GEM and treatment group and survival analyses of mice in select groups. Log rank test. FIG. 6D. TCGA data set analysis evaluating FOXP3, GADH, and ACTA2 (αSMA) transcript levels in tumors with high (IL-6 HI) and low (IL-6 LO) IL-6 transcript levels. Unpaired two-tailed t test. * P<0.05, *** P<0.005, ns: not significant. Gem: gemcitabine, aIL-6: anti-IL-6 antibodies, CP: anti-CTLA-4 and anti-PD1 antibodies. See Table 1 for mouse GEM nomenclature.

FIGS. 7A-B. Gating strategy and control used to define the gates shown in FIGS. 1E-F. Tumor cells gating strategy and FAP isotype control (FIG. 7A), unstained spleen cells used as a negative control for the endogenous fluorescence (αSMA-RFP and YFP) gating (FIG. 7B).

FIGS. 8A-E. FIG. 8A. Survival curve of PKP mice with and without αSMA cell depletion (PKP control are PKP mice without the αSMA-TK transgene and administered with GCV). The arrow indicates when GCV treatment was initiated. The lines that falls to 0% survival at about 75 days represents PKP αSMA depleted. Log rank test. FIG. 8B. Bar graph depicting the histological features of the indicated groups based on H&E staining of pancreas tumor sections of PKP GEM with and without αSMA⁺ cell depletion, aged match or end point. Control include PKP mice harboring the transgene without GCV or/and PKT mice administered GCV and without the transgene. Age matched-control, n=9; Control moribund (experimental endpoint), n=11, αSMA-TK, n=12 mice. Statistical significance was evaluated using two-way ANOVA. FIG. 8C. Tumor burden expressed as a percentage of tumor weight to body weight. Control, n=8; FAP-TK, n=9 mice. Unpaired two-tailed t test. FIG. 8D. Flow cytometry evaluation of FAP⁺ cells in tumors of the indicated mice and graphical representation (one mouse per group). FIG. 8E. Tumor histopathologic score. Control, n=4; FAP-TK, n=4 mice. Mean+/−standard deviation, Mann-Whitney test. Unless otherwise stated, the data are presented as the mean+/−standard error of the mean. * P<0.05, *** P<0.005, ****, P<0.001, ns: not significant. GEM: genetically engineered mice, GCV: ganciclovir, see Table 1 for mouse GEM nomenclature.

FIGS. 9A-E. FIG. 9A. Body weight measurement over time in non-tumor bearing mice given GCV. WT (bottom line): wildtype littermate control, n=3; FAP-TK (top line), n=4 mice. FIG. 9B. Pancreas, spleen, quadriceps muscle (QM), and gastroecmius muscle (GM) weight at end point. WT: wildtype littermate control, n=3; FAP-TK, n=4 mice. FIG. 9C. Flow cytometry for FAP⁺ cells in the spleen of PKT mice. FIG. 9D. Evaluation using flow cytometry of αSMA-RFP⁺ (n=2 mice) and FAP immunolabeled cells (n=3 mice) in the bone marrow of a non-tumor bearing mouse. FIG. 9E. Increase in the frequency of FAP⁺ cells in the bone marrow of tumor bearing mice (PKT, n=3) compared to non-tumor bearing control mice (n=6). The mean+/−standard error of the mean is depicted, unpaired two-tailed t test, * P<0.05, ns: not significant. GCV: ganciclovir, see Table 1 for mouse nomenclature.

FIGS. 10A-C. FIG. 10A. Representative H&E stained, CK19 and αSMA immunolabeled pancreas, lung, and liver sections from KPPF GEM, at the listed time points. Scale bar: 100 μm. FIG. 10B. Representative H&E stained pancreas sections from KPPC GEM, at the listed time points. Scale bar: 100 μm. FIG. 10C. Representative immunofluorescent capture of GFP, RFP, YFP and CFP and nuclei in the pancreas tumor of KPPF;αSMA-Cre;R26^{Confetti} GEM. Scale bar: 20 μm. GEM: genetically engineered mice, wks: weeks, ADM: Acinar to ductal metaplasia, see Table 1 for mouse GEM nomenclature.

FIGS. 11A-F. FIG. 11A. Schematic representation of tumor derived cancer cells and fibroblasts harvested from KPPF;αSMA-Cre;R26^{Dual} GEM. FIG. 11B. Expression of IL-1β in the tumors of listed GEM by qPCR. From left to right, the bars represent KPPF, KPPF;IL-6^{smaKO}, and KPPF;IL-6^{-/-}. n=mice per group. FIGS. 11C-E. Electrophoretic migration of PCR products of the DNA purified from the listed organs and GEM. Product detection confirm specific deletion of IL-6 by gene recombination in the expected lanes. FIG. 11F. Quantitation of co-localization of immunolabeling for FAP and αSMA in tumors from KPPF;αSMA-Cre;R26^{Dual} GEM. n=4 mice. The mean+/−standard error of the mean is presented, ns: not significant, GEM: genetically engineered mice, qPCR: quantitative PCR, see Table 1 for mouse GEM nomenclature.

FIGS. 12A-C. FIG. 12A. Tumor burden in the listed GEM. From left to right, the bars represent KPPF, KPPF;IL-6^{smaKO}, and KPPF;IL-6^{-/-}. FIG. 12B. Incidence of metastases in the indicated GEM. FIG. 12C. Quantitation of the histopathological features of H&E stained pancreas section from the listed GEM. Within each column, the sections represent, from top to bottom, PanIN and Normal. GEM: genetically engineered mice, ns: not significant, see Table 1 for mouse GEM nomenclature.

FIGS. 13A-C. FIG. 13A. Representative H&E stained and CK19 immunolabeled pancreas of KPF mice at the listed time point of disease progression, and H&E stained sections of liver and lung metastasis (black arrow). Scale bar: 100 μm. FIG. 13B. Representative H&E stained sections of pancreas of the listed GEM. Scale bar: 100 μm. FIG. 13C. Survival of the listed GEM, see FIG. 6C. The line that intersects with the x-axis at just under 400 days represents KPF. Log rank test. GEM: genetically engineered mice, ns: not significant, see Table 1 for mouse GEM nomenclature.

FIGS. 14A-D. FIG. 14A. Survival of the listed GEM, see FIG. 6C. Log rank test. FIG. 14B. Quantitation of the histopathological features of H&E stained section of the pancreas of the listed GEM at moribund endpoint. Within each column, the sections represent, from top to bottom, necrosis, poor, well, PanIN, and Normal. KPPF Gem, n=6; KPPF; IL-6^{smaKO} Gem, n=5 mice. Two-way ANOVA. FIG. 14C. Tumor burden in the indicated GEM. KPPF Gem (left column), n=8; KPPF; IL-6^{smaKO} Gem (right column), n=11 mice. Unpaired two-tailed t test. FIG. 14D. Quantitation of the percent αSMA⁺ area from αSMA immunolabeled section of the pancreas of the listed GEM. n=5 mice per group, one-way ANOVA. * P<0.05, ** P<0.01, ***, P<0.005, ****, P<0.001, ns: not significant. Data is presented as the mean+/−standard error of the mean. GEM: genetically engineered mice, Gem: gemcitabine, see Table 1 for mouse GEM nomenclature.

FIGS. 15A-B. FIG. 15A. Quantification of percent stained area of pancreas sections from the listed GEMs, immunolabeled for phospho-ERK1/2 and phospho-Akt. n=5 mice per group, one-way ANOVA. FIG. 15B. Quantification of percent stained area for pancreas sections from the listed GEMs, immunolabeled for CD31, Ki67, cleaved caspase 3, and stained for MTS. n=5 mice per group, one-way. ANOVA. * P<0.05, ** P<0.01, ***, P<0.005, ****, P<0.001, ns: not significant. GEM: genetically engineered mice, Gem: gemcitabine, see Table 1 for mouse GEM nomenclature.

FIGS. 16A-D. Gating strategies for flow cytometry analyses of tumors (FIG. 16A) and spleen (FIG. 16B) for immunotyping analyses. Gating strategies for flow cytometry analyses of tumors for the T cell panel (FIG. 16C) and myeloid cell panel (FIG. 16D).

FIGS. 17A-C. Additional immunotyping results for the listed immune cells in the indicated GEMs, evaluating tumor (FIG. 17A), spleen (FIG. 17B), and peripheral blood (FIG. 17C).

### DETAILED DESCRIPTION

The desmoplastic reaction in pancreatic ductal adenocarcinomas (PDAC) involves a significant accumulation of immune cells and fibroblasts. Fibroblasts are mesenchymal cells that contribute to tissue repair and regeneration and accumulate in tumors as part of the host response to cancer. The origin and functional diversity of such carcinoma associated fibroblasts (CAFs) remains largely unknown. αSMA+ cells are a dominant CAF population in PDAC with tumor restraining properties (TS-CAFs), as opposed to FAP+ CAFs, which demonstrate tumor promoting activity (TP-CAFs). While TS-CAFs predominantly modulate extracellular matrix (ECM) production, facilitate cell-ECM adhesion, and regulate adaptive immunity, TP-CAFs exhibit a lineage that is skewed towards a pro-inflammatory, chemokine secreting phenotype and exhibit expression of unique genes that can serve as diagnostic and therapeutic targets to restrain TP-CAFs. Further, CAFs share distinct gene expression profiles characteristic of lymphocytic and myeloid lineages. Although αSMA⁺ CAFs-derived interleukin-6 (IL-6) does not impact PDAC progression, it contributes to chemoresistance and attenuates the potential of immune checkpoint blockade therapy. Specific deletion of IL-6 from αSMA⁺ CAFs enhanced sensitivity to chemotherapy and checkpoint blockade therapy. Collectively, these studies identify a complex network of functionally heterogeneous fibroblasts during PDAC progression with significant therapeutic implication. As such, provided herein are CAF targets for controlling tumor growth.

The results using genetic moue models identify that PDAC CAFs are a heterogeneous population with αSMA⁺ CAFs emerging as a dominant population in PDAC. These studies entirely refrained from the use of cell culture systems to arrive at this conclusion, as an effort to make the study more relevant to human PDAC biology. αSMA⁺ CAFs and FAP⁺ CAFs have different functions in PDAC progression (Feig et al., 2013; Lo et al., 2017; Kraman et al., 2010). Interesting, FAP⁺ CAFs represent a bone marrow-derived progenitor that can give rise to αSMA⁺ CAFs. Surprisingly, the identities of αSMA⁺ CAFs and FAP⁺ CAFs, as defined by scRNA-Seq, reflect distinct immune cell-like transcriptomes. The αSMA⁺ CAFs contain a subset of collagen producing and remodeling cells, as well as cells that displayed a macrophage-like, and a T cell-like transcriptome. In contrast, the FAP⁺ CAFs comprised a subset of cells with a neutrophil-like, and a B cell-like transcriptome. A subset of αSMA⁺ CAFs and FAP⁺ CAFs showed a monocyte and dendritic cell-like transcriptome. These findings are intriguing because they highlight a possible overlap between mesenchymal and hematopoietic cell lineages. A rich cytokine milieu in the PDAC tumor microenvironment regulates immune cell recruitment and phenotype maturation, and exposure of CAFs to this milieu may result in an induction of immune cell associated transcripts. It is possible that certain CAFs may foster a feedforward signaling loop to manipulate the intra-tumoral immune response. In the absence of specific CAF population, the differentiation/activation of certain immune cells may be hindered or limited in PDAC. Alternatively, the CAFs may carry out immune cell functions in the tumors.

αSMA⁺ CAFs-derived IL-6 confers chemoresistance and negatively regulates T cells in the tumor microenvironment. In this regard, in organoid-based study, a subset of CAFs were highlighted to represent an immunomodulatory phenotype that included IL-6 production (Ohlund et al., 2017). While, in sum totality, the αSMA⁺ CAFs emerged as tumor restraining (tumor suppressing or TS-CAFs) in PDAC progression, in the context of gemcitabine therapy stress, cancer cells ‘utilize’ the IL-6 produced by αSMA⁺ CAFs to promote their survival. It is conceivable that the IL-6 produced by the αSMA⁺ CAFs is for self-preservation purposes in non-treatment conditions, but is also utilized by cancer cells for induction of pro-survival signaling pathways realized in the context of resistance to gemcitabine treatment. Previous studies have reported that IL-6 signaling confers pro-survival signals to cancer cells via the JAK/STAT signaling pathway in the context of chemotherapy (Wormann et al., 2016; Nagathihalli et al., 2016). A significant polarization of the PDAC immune microenvironment has been observed upon deletion of αSMA⁺ CAF produced IL-6; however, such changes in Teff and Treg frequencies did not impact PDAC progression Immune checkpoint blockade therapy was ineffective when combined with gemcitabine; however, such combinatorial benefit was realized when combined with suppression of IL-6 signaling, indicating that IL-6 is a critical suppressor of immune checkpoint blockade therapy in PDAC. Suppression of IL-6 likely favored emergence of effector T cells that, when combined with the cell death and possibly neo-antigen generation induced by gemcitabine, could have augmented the efficacy of immune checkpoint blockade.

### I. Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

B. General Methods

It will be understood that monoclonal antibodies binding to proteins highly expressed by TP-CAFs will have several applications. These include the production of diagnostic kits for use in detecting and diagnosing cancer, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce cancer-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art. HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily Fusion methods using Sendai virus have been described, and those using polyethylene glycol (PEG), such as 37% (v/v) PEG. The use of electrically induced fusion methods also is appropriate and there are processes for better efficiency. Fusion procedures usually produce viable hybrids at low frequencies, about 1×10⁻⁶ to 1×10⁻⁸, but with optimized procedures one can achieve fusion efficiencies close to 1 in 200. However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

C. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti-CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1----6) Dextran Increases Its Affinity For Antigen,” J. Exp. Med. 168(3): 1099-1109; Tao, M. H. et al. (1989) “Studies Of Aglycosylated Chimeric Mouse-Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

D. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for treatment of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described. High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life, including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH₂ domain (according to the IMGT unique numbering for C-domain) were substituted with alanine residues. This modification, also known as “LALA” mutation, abolishes antibody binding to FcγRI, FcγRII and FcγRIIIa. The variant and unmodified recombinant mAbs were compared for their capacity to neutralize and enhance infection by the four dengue virus serotypes. LALA variants retained the same neutralizing activity as unmodified mAb, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication US 2003/0003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection; however it has become clear that many human naturally occurring antibodies with autoreactive properties persist in adult mature repertoires. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

E. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane. The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

F. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an antigen-specific arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess an antigen-binding arm and an arm that binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′).sub.2 bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998)). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

G. Chimeric Antigen Receptors

Chimeric antigen receptor molecules are recombinant fusion protein and are distinguished by their ability to both bind antigen and transduce activation signals via immunoreceptor activation motifs (ITAMs) present in their cytoplasmic tails. Receptor constructs utilizing an antigen-binding moiety (for example, generated from single chain antibodies (scFv) afford the additional advantage of being “universal” in that they bind native antigen on the target cell surface in an HLA-independent fashion.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell or an NK cell.

Embodiments of the CARs described herein include nucleic acids encoding an antigen-specific chimeric antigen receptor (CAR) polypeptide, including a comprising an intracellular signaling domain, a transmembrane domain, and an extracellular domain comprising one or more signaling motifs. In certain embodiments, the CAR may recognize an epitope comprised of the shared space between one or more antigens. In some embodiments, the chimeric antigen receptor comprises: a) an intracellular signaling domain, b) a transmembrane domain, and c) an extracellular domain comprising an antigen binding domain. Optionally, a CAR can comprise a hinge domain positioned between the transmembrane domain and the antigen binding domain. In certain aspects, a CAR of the embodiments further comprises a signal peptide that directs expression of the CAR to the cell surface. For example, in some aspects, a CAR can comprise a signal peptide from GM-CSF.

In certain embodiments, the CAR can also be co-expressed with a membrane-bound cytokine to improve persistence when there is a low amount of tumor-associated antigen. For example, CAR can be co-expressed with membrane-bound IL-15.

Depending on the arrangement of the domains of the CAR and the specific sequences used in the domains, immune effector cells expressing the CAR may have different levels activity against target cells. In some aspects, different CAR sequences may be introduced into immune effector cells to generate engineered cells, the engineered cells selected for elevated SRC and the selected cells tested for activity to identify the CAR constructs predicted to have the greatest therapeutic efficacy.

1. Antigen Binding Domain

In certain embodiments, an antigen binding domain can comprise complementary determining regions of a monoclonal antibody, variable regions of a monoclonal antibody, and/or antigen binding fragments thereof. In another embodiment, that specificity is derived from a peptide (e.g., cytokine) that binds to a receptor. A “complementarity determining region (CDR)” is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen—each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions.

It is contemplated that the CAR nucleic acids, in particular the scFv sequences are human genes to enhance cellular immunotherapy for human patients. In a specific embodiment, there is provided a full length CAR cDNA or coding region. The antigen binding regions or domains can comprise a fragment of the VH and VL chains of a single-chain variable fragment (scFv) derived from a particular mouse, or human or humanized monoclonal antibody. The fragment can also be any number of different antigen binding domains of an antigen-specific antibody. In a more specific embodiment, the fragment is an antigen-specific scFv encoded by a sequence that is optimized for human codon usage for expression in human cells. In certain aspects, VH and VL domains of a CAR are separated by a linker sequence, such as a Whitlow linker. CAR constructs that may be modified or used according to the embodiments are also provided in International (PCT) Patent Publication No. WO/2015/123642, incorporated herein by reference.

As previously described, the prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to an antigen of interest, such as tumor associated antigen. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, it is shown that a CAR may be engineered using VH and VL chains derived from different mAbs to generate a panel of CAR+ T cells. The antigen binding domain of a CAR can contain any combination of the LCDR1-3 sequences of a first antibody and the HCDR1-3 sequences of a second antibody.

2. Hinge Domain

In certain aspects, a CAR polypeptide of the embodiments can include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-gene modified T cells. In some aspects, the hinge domain comprises a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor. In certain aspects, the hinge domain (and/or the CAR) does not comprise a wild type human IgG4 CH2 and CH3 sequence.

In some cases the CAR hinge domain could be derived from human immunoglobulin (Ig) constant region or a portion thereof including the Ig hinge, or from human CD8 a transmembrane domain and CD8a-hinge region. In one aspect, the CAR hinge domain can comprise a hinge-CH₂-CH₃ region of antibody isotype IgG4. In some aspects, point mutations could be introduced in antibody heavy chain CH₂ domain to reduce glycosylation and non-specific Fc gamma receptor binding of CAR-T cells or any other CAR-modified cells.

In certain aspects, a CAR hinge domain of the embodiments comprises an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. In some aspects, a CAR hinge domain comprises an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. In further aspects, a CAR hinge domain can comprise an IgG4-Fc domain having an amino acid substitution at position L235 for an amino acid that is hydrophilic, such as R, H, K, D, E, S, T, N or Q or that has similar properties to an “E” such as D. In certain aspects, a CAR hinge domain can comprise an IgG4-Fc domain having an amino acid substitution at position N297 for an amino acid that has similar properties to a “Q” such as S or T.

In certain specific aspects, the hinge domain comprises a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain or an engineered hinge domain.

3. Transmembrane Domain

The antigen-specific extracellular domain and the intracellular signaling-domain may be linked by a transmembrane domain Polypeptide sequences that can be used as part of transmembrane domain include, without limitation, the human CD4 transmembrane domain, the human CD28 transmembrane domain, the transmembrane human CD3ζ domain, or a cysteine mutated human CD3ζ domain, or other transmembrane domains from other human transmembrane signaling proteins, such as CD16 and CD8 and erythropoietin receptor. In some aspects, for example, the transmembrane domain comprises a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8a transmembrane domain), both incorporated herein by reference. Transmembrane regions of particular use in this invention may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

4. Intracellular Signaling Domain

The intracellular signaling domain of the chimeric antigen receptor of the embodiments is responsible for activation of at least one of the normal effector functions of the immune cell engineered to express a chimeric antigen receptor. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus the term “intracellular signaling domain” refers to the portion of a protein that transduces the effector function signal and directs the cell to perform a specialized function. In some aspects, the intracellular signaling domain is derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB, DAP-10, OX40, and combinations thereof, as well as other similar molecules and fragments. Intracellular signaling portions of other members of the families of activating proteins can be used. While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. In a preferred embodiment, the human CD3ζ intracellular domain is used as the intracellular signaling domain for a CAR of the embodiments.

In specific embodiments, intracellular receptor signaling domains in the CAR include those of the T cell antigen receptor complex, such as the ζ chain of CD3, also Fcγ RIII costimulatory signaling domains, CD28, CD27, DAP10, CD137, OX40, CD2, alone or in a series with CD3ζ, for example. In specific embodiments, the intracellular domain (which may be referred to as the cytoplasmic domain) comprises part or all of one or more of TCRζ chain, CD28, CD27, OX40/CD134, 4-1BB/CD137, FcεRIγ, ICOS/CD278, IL-2Rβ/CD122, IL-2Rα/CD132, DAP10, DAP12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example the CD28 and 4-1BB can be combined in a CAR construct.

In some embodiments, the CAR comprises additional other costimulatory domains. Other costimulatory domains can include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of T cells and could help improve in vivo persistence and the therapeutic success of the adoptive immunotherapy.

In certain specific aspects, the intracellular signaling domain comprises a sequence 85%, 90%, 91%, 92%, 93%, 94%, 95%, 96%, 97%, 98%, 99% or 100% identical to a CD3ζ intracellular domain, a CD28 intracellular domain, a CD137 intracellular domain, or a domain comprising a CD28 intracellular domain fused to the 4-1BB intracellular domain.

H. ADCs

Antibody Drug Conjugates or ADCs are a new class of highly potent biopharmaceutical drugs designed as a targeted therapy for the treatment of people with disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the diseased cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs cellular replication. Due to this targeting, ideally the drug has lower side effects and gives a wider therapeutic window than other agents.

A stable link between the antibody and cytotoxic agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

I. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

J. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. One means of delivery comprises the use of lipid-based nanoparticles, or exosomes, as taught in U.S. Pat. Appln. Publn. 2018/0177727, which is incorporated by reference here in its entirety. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.).

K. Purification

The antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

L. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵ sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2- and 8-azido nucleotides have also been used to map nucleotide binding domains of purified proteins and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature. This approach has been reported to produce diagnostically and therapeutically promising antibodies which are currently in clinical evaluation.

### II. Methods of Treatment

Certain aspects of the present embodiments can be used to prevent or treat a disease or disorder associated with the presence of TP-CAFs, such as pancreatic ductal adenocarcinoma. Functioning of TP-CAFs may be reduced by any suitable drugs. For example, such substances could be an anti-TP-CAFs antibody or chimeric antigen receptor. In addition, the present embodiments can be used to treat a cancer that has previously been resistant to immune checkpoint blockade therapy by administering IL-6 signaling inhibitors in combination with immune checkpoint blockade therapy, and also optionally in combination with standard of care chemotherapy.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that targets TP-CAFs either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease, such as a cancer. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, a neurodegenerative disease, and/or a genetic disorder).

A. Formulation and Administration

The present disclosure provides pharmaceutical compositions comprising antibodies that selectively target TP-CAFs. Such compositions comprise a prophylactically or therapeutically effective amount of an antibody or a fragment thereof and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Passive transfer of antibodies generally will involve the use of intravenous or intramuscular injections. The forms of antibody can be as monoclonal antibodies (MAb). Such immunity generally lasts for only a short period of time, and there is also a potential risk for hypersensitivity reactions, and serum sickness, especially from gamma globulin of non-human origin. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Kits and Diagnostics

In various aspects of the embodiments, a kit is envisioned containing therapeutic agents and/or other therapeutic and delivery agents. The present embodiments contemplate a kit for preparing and/or administering a therapy of the embodiments. The kit may comprise one or more sealed vials containing any of the pharmaceutical compositions of the present embodiments. The kit may include, for example, at least one anti-TP-CAFs antibody as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

C. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

D. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have a cytotoxic effect.

E. Combination Therapy

In certain embodiments, the compositions and methods of the present embodiments involve an antibody or an antibody fragment against TP-CAFs to inhibit their activity, in combination with a second or additional therapy, such as chemotherapy or immunotherapy. Such therapy can be applied in the treatment of any disease that is associated with TP-CAFs. For example, the disease may be a cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

A therapeutic antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an antibody therapy is “A” and an anti-cancer therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169); cytokine therapy, e.g., interferons α, β, and γ, IL-1, GM-CSF, and TNF; gene therapy, e.g., TNF, IL-1, IL-2, and p53 (U.S. Pat. Nos. 5,830,880 and 5,846,945); and monoclonal antibodies, e.g., anti-CD20, anti-ganglioside GM2, and anti-p185 (U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, may be antibodies, such as human antibodies (e.g., International Patent Publication WO2015016718, incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Mice. All acronyms designating specific genetically engineered mice (GEM) are listed in Table 1. FSF-Kras^{G12D/+} (Schonhuber et al., 2014), Pdx1-Flp (Schonhuber et al., 2014), Trp53^{frt/+} (Lee et al., 2012), LSL-Kras^{G12D/+} (Hingorani et al., 2005), Pdx1-Cre (Hingorani et al., 2005), αSMA-Cre (LeBleu et al., 2013), αSMA-RFP (LeBleu et al., 2013), Rosa26-loxP-Stop-loxP-YFP (Ozdemir et al., 2014), Tgfbr2^{loxP/loxP} (Ijichi et al., 2006), and IL-6^{loxP/loxP} (Quintana et al., 2013) mouse strains were previously documented. The Rosa26-CAG-loxP-frt-Stop-frt-FireflyLuc-EGFP-loxP-RenillaLuc-tdTomato (referred to as R26^{Dual}), FSF-Kras^{G12D/+}, Pdx1-Flp, and Trp53^{frt/+} strains were kindly provided by D. Saur, and the R26R-Brainbow2.1/Confetti (referred to as R26^{Confetti}) strain was purchased from Jackson Laboratory (Stock 013731). The IL-6^{-/-} strain was generated by crossing the IL-6^{loxP/loxP} strain with CMV-Cre strain (Jackson Laboratory; Stock 006054). The FAP-TK transgenic strain was newly generated: a 5 kb sequence flanking the FAP promoter and partial Exon 1 (Ex1) was cloned into pORF-HSV1-TK vector (Invivogen) using NotI and AgeI. The sequence-confirmed FAP-TK construct was released from the vector using NotI and SwaI before purification and injection into fertilized eggs. The transgenic mice were generated on the C57Bl/6 genetic background. These mice were bred onto PDAC GEM or implanted orthotopically with 689KPC cancer cells, as previously described (Kamerkar et al., 2017). Mice were maintained on a mixed genetic background and both male and female mice were evaluated. Mice were given gemcitabine (G-4177, LC Laboratories) intraperitoneally (i.p.) twice per week at 40 mg/kg of body weight. Gemcitabine treatment was initiated at 35 days of age. Ganciclovir (GCV; sud-gcv, Invivogen) was administered i.p. daily at 50 mg/kg of body weight (approximating 1.5 mg per 25 g mouse). GCV was administered to PKT mice at 28 to 30 days of age, and to PKP mice at 50-51 days of age. Some of the tissues analyzed from the PKT;αSMA-TK GEM were from mice that were previously published (Ozdemir et al., 2014). Control groups received phosphate buffer saline instead of GCV or were not injected. In the orthoptic tumor model (689KPC), GCV was administered 15 days following tumor implantation and mice euthanized at 40 days following tumor implantation. Anti-IL-6 antibodies (MP5-20F3; BioXCell) were administered i.p. at a dose of 200 μg/mouse twice per week. Treatment was initiated at 35 days of age. Anti-CTLA-4 (BE0131, clone 9H10, BioXCell) and anti-PD1 (BE0273, 29F.1A12, BioXCell) antibodies were administered i.p. at a dose of 100 μg/mouse each, for a total of 3 injections 3 days apart (initiated at 35 days of age). All mice were housed under standard housing conditions. Investigators were not blinded to group allocation but were blinded for the histological assessment of phenotypic outcome. No randomization method was used and no animal was excluded from the analysis. The experimental endpoint was defined when the animals developed significant signs of illness leading to their death or requiring euthanasia.

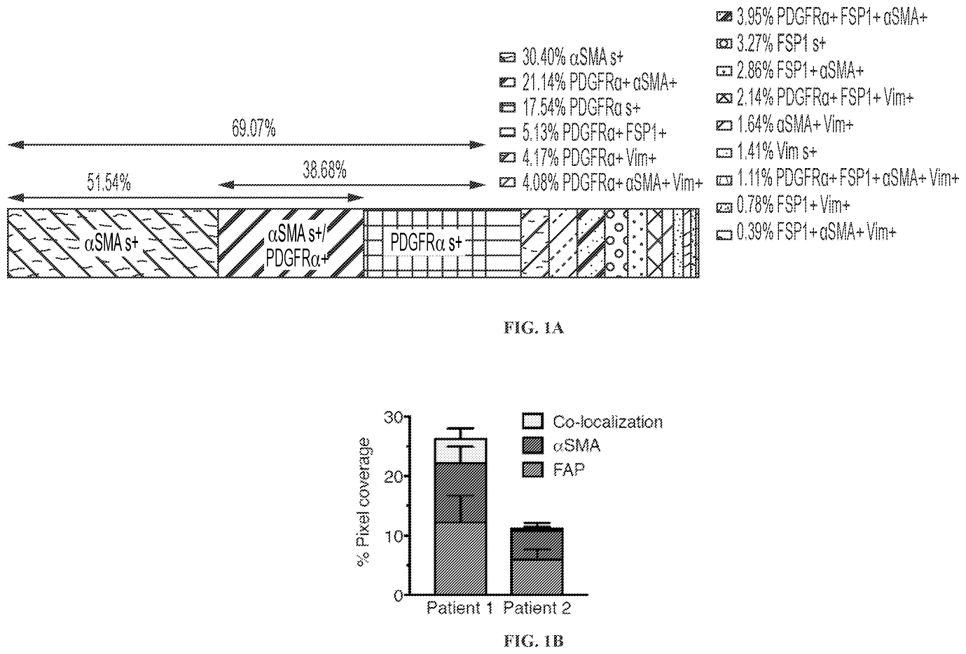

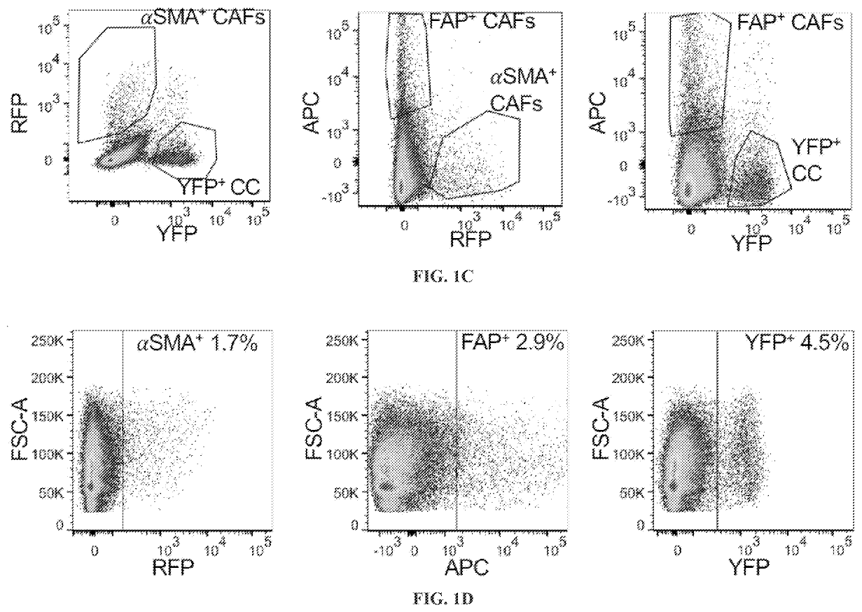

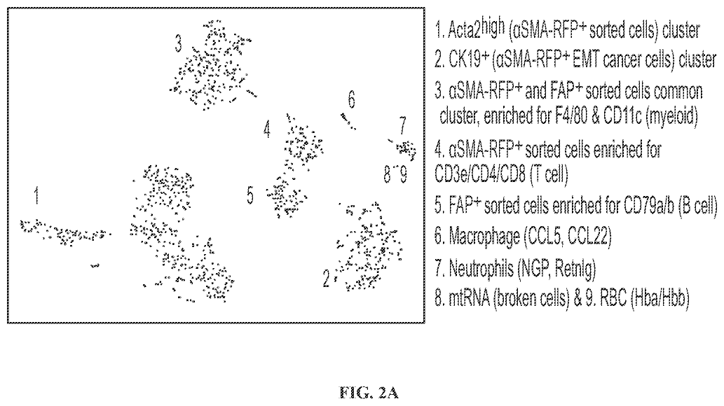

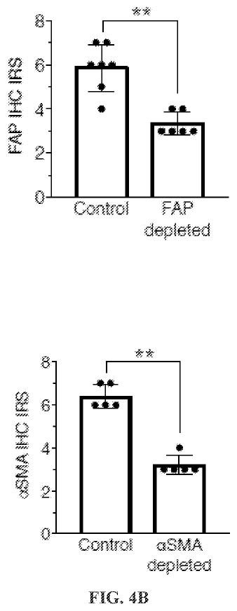

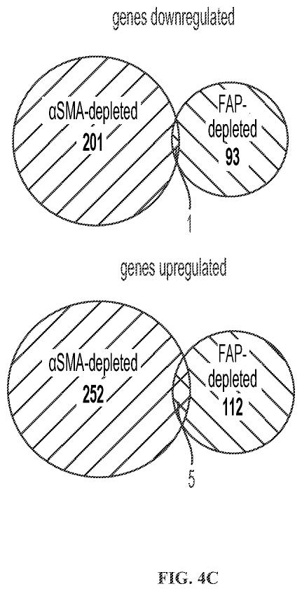

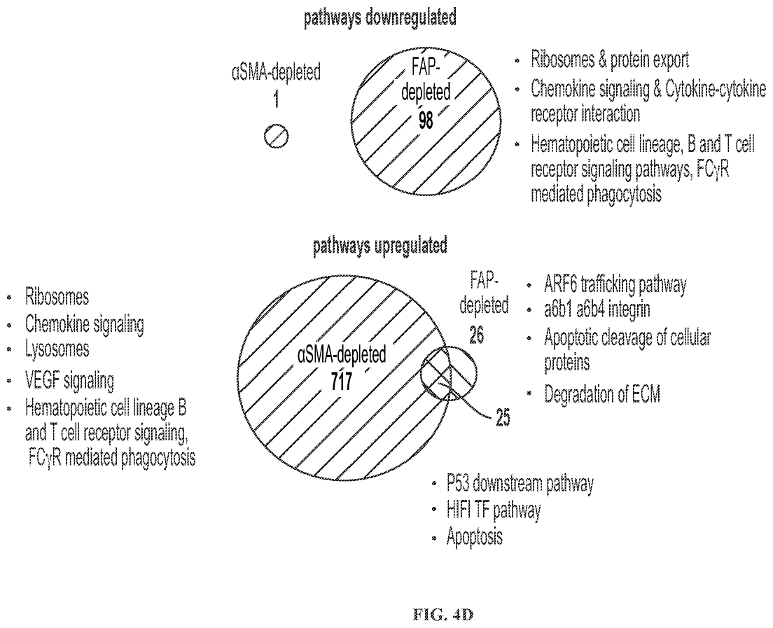

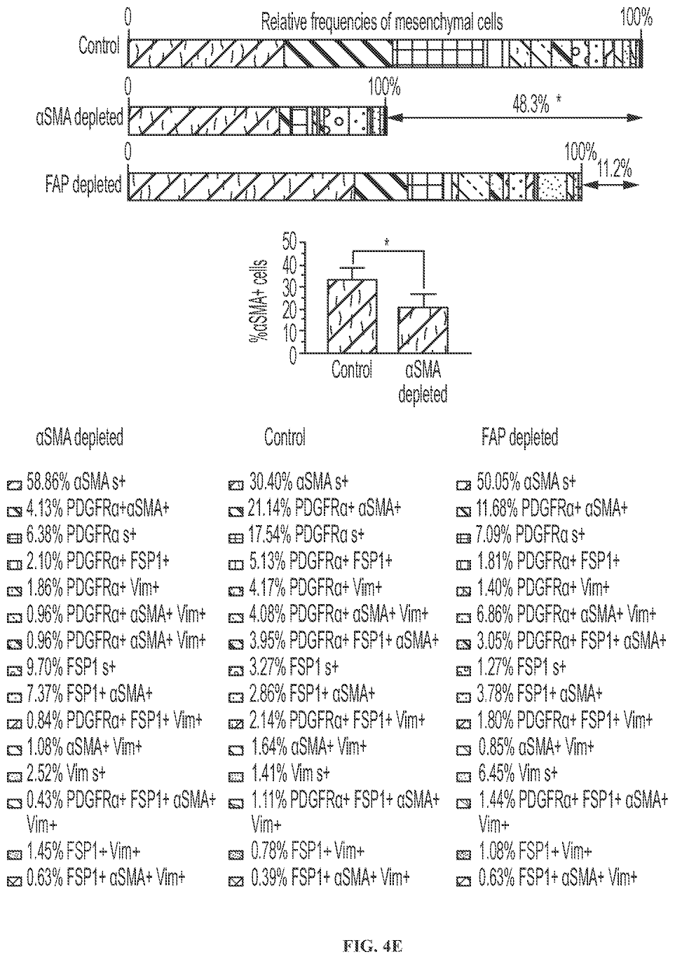

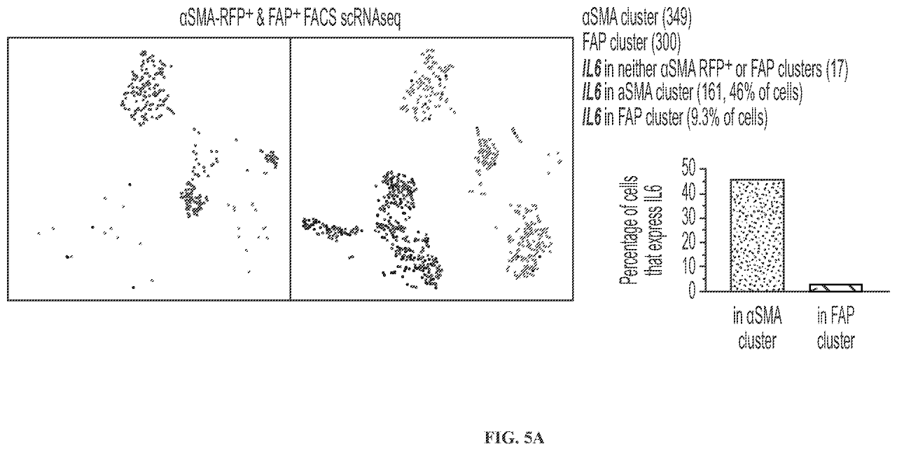

Multispectral imaging of multiplex stained tissue sections. The multiplex staining procedures, spectral unmixing, and cell segmentation using Nuance and inForm imaging software were previously published (Carstens et al., 2017). Antibody concentrations used for the multiplex staining can be found in Table 1. Multiplex stained slides were imaged with the Vectra Multispectral Imaging System, using Vectra software version 3.0.3 (Perkin Elmer). Each tissue section was scanned in its entirety using a 4× objective, and up to 80 regions (at 20×) were selected for multispectral imaging using the Phenochart software (Perkin Elmer). Each multiplex field was scanned every 10 nm of the emission light spectrum across the range of each emission filter cube. Filter cubes used for multispectral imaging were DAPI (440-600 nm), FITC (520 nm-680 nm), Cy3 (570-690 nm), Texas Red (580-700 nm). and Cy5 (680-720 nm). Multispectral images from single marker stained slides with the corresponding fluorophores were used to generate a spectral library using the Nuance Image Analysis software (Perkin Elmer). The library contained the emitting spectral peaks of all fluorophores and was used to unmix each multispectral image (spectral unmixing) to its individual six components by using the inForm 2.2 image analysis software. All spectrally unmixed image cubes were subsequently segmented into individual cells based on the nuclear DAPI counterstain. The cell segmentation information including the individual cell expression levels of all markers was then processed using an R algorithm, with marker positivity being identified for each cell based on a threshold of staining positivity previously determined using the inForm software. Thresholds of detection for the different markers were adjusted across different cohorts in order to ensure consistent capture of positive signal across all controls. All images in each cohort were processed using the same thresholds of staining positivity. Mesenchymal cell composition in PKT combined control tumors (FIGS. 1A and 4E) include FAP-TK^{control}, n=7, and αSMA-TK^{control}, n=4. The percent differences listed in FIG. 4E are comparisons between depleted and respective control groups, and these results were similar if the comparison was carried out using the combined controls. Antibody sources and dilutions are detailed in Table 2.

Immunofluorescent labeling and immunohistochemistry. All antibodies, sources, and dilutions are listed in Table 2. For EGFP/tdTomato visualization, tissues from the KPPF;αSMA-Cre;R26^{Dual} and KPPF;αSMA-Cre;R26^{Confetti} mice were fixed in 4% paraformaldehyde overnight at 4° C. and equilibrated in 30% sucrose overnight at 4° C. Tissues were then embedded in O.C.T. compound (TissueTek) and processed for 5-μm-thick frozen sections. Sections were blocked for 1 h with 4% cold water fish gelatin (Aurion) and immunostained overnight at 4° C. with anti-αSMA antibody (followed by AlexaFluor647 secondary antibody) or FAP antibody followed by AlexaFluor405 secondary antibody). Slides were then mounted with Vectashield Mounting Medium (Vector Laboratories) to a glass coverslip and visualized under the LSM800 confocal laser scanning microscope and ZEN software (Zeiss). For visualization of endogenous EYFP and RFP fluorescence, tissue from PKTY;αSMA-RFP mice were fixed in 4% paraformaldehyde overnight at 4° C. and equilibrated in 30% sucrose overnight at 4° C. Tissues were then embedded in O.C.T. compound (TissueTek) and processed for 5 μm-thick sections. Sections were fixed in cold acetone, mounted with DAPI mounting media, and visualized with a laser scanning confocal microscope (Olympus FV1000) with a 20×0.85 NA UPLSAPO objective lens using the 405, 488, and 559 nm lasers Images were acquired with a FLUOVIEW FV100 software version 4.0.3.4 (Olympus).

Formalin-fixed, paraffin-embedded (FFPE) sections were processed for immunohistochemical (IHC) staining as previously described (Zheng et al., 2015), after Citrate based antigen retrieval (pH=6). For FAP staining in mouse FFPE sections citrate-based antigen retrieval was performed at pH 7.4. Staining for αSMA was performed with M.O.M. kit (Vector Laboratories) following the manufacturer's instructions. For all other staining, sections were incubated with biotinylated goat anti-rabbit and streptavidin HRP (Biocare Medical), each for 10 min. For all staining, counterstaining with hematoxylin was performed and DAB positivity was examined in ten visual fields at 200× magnification. Cleaved caspase-3 staining was quantified by counting the number of cells that exhibited positive nuclei per 400× visual field. The images were quantified for positive area using NIH ImageJ analysis software (αSMA, CD31, CK19, cleaved caspase-3, Ki67, MTS, phospho-Akt, phospho-ERK1/2, phospho-Stat3). For αSMA and FAP double immunofluorescence in human FFPE sections, secondary anti-sheep Alexa Fluor 488 and anti-mouse Alexa Fluor 534 antibodies were incubated for 30 min at RT. For αSMA and FAP IHC on FAP-TK and control tumor sections, immunoreactive score (IRS) were obtained from the sum of distribution and intensity scores for each section, established on a scale of 1 to 4 (Meyerholz & Beck, 2018).

Flow Cytometry. For analysis of YFP, αSMA-RFP, and FAP immunolabeled cells from the tumors of PKTY;αSMA-RFP mice, the tumors were minced and digested in collagenase IV (4 mg/mL) and dispase (4 mg/mL) in DMEM media for 1 hour at 37° C. Digested tissues were then filtered through a 70 μm mesh followed by a 40 μm mesh, centrifuged, and incubated in ACK lysis buffer for 3 minutes at room temperature. FAP and its corresponding isotype antibody were conjugated with Zenon Alexa Fluor 647 Rabbit IgG labeling kit according to manufacturer's instructions. Samples were stained with antibody and fixable viability dye eFluor 780 in FACS buffer for 30 minutes on ice followed by washing prior to analysis on a BD LSR Fortessa X20. For sorting experiments, samples were analyzed and sorted on a BD FACS Aria. Bone marrow, flushed from the long bones, and spleen of wild type, αSMA-RFP, and FAP-TK mice were also immunolabeled for FAP. Prior to staining, spleen was minced and filtered through a 40 μm mesh and both spleen and bone marrow were incubated in ACK lysis buffed for 3 minutes at room temperature. Unstained and single-stained samples were used for compensation controls. Details on the antibodies, sources, and dilutions are listed in Table 2.

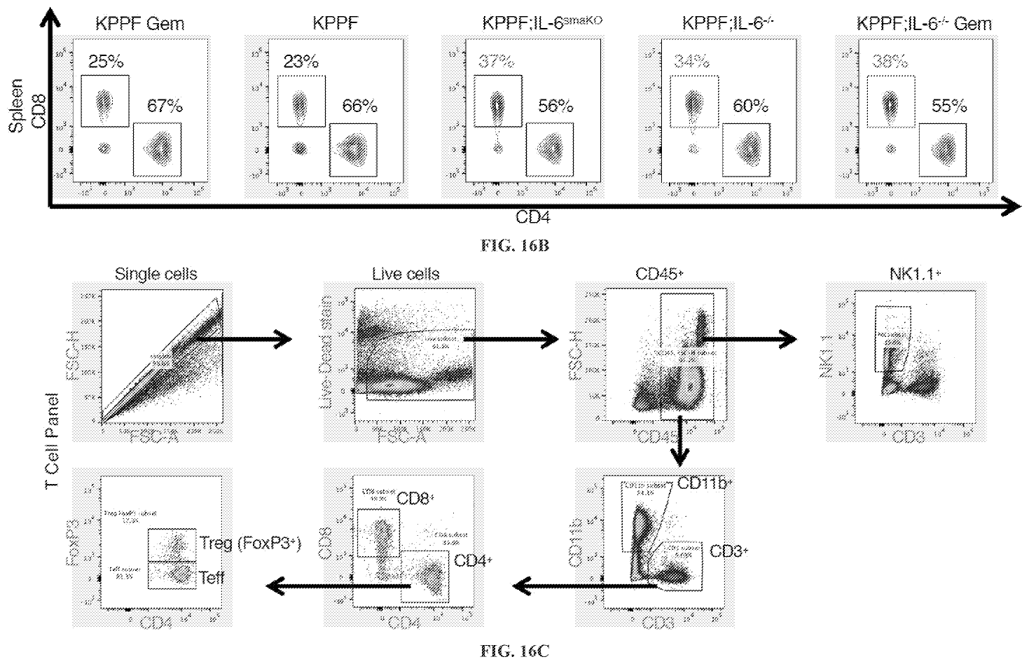

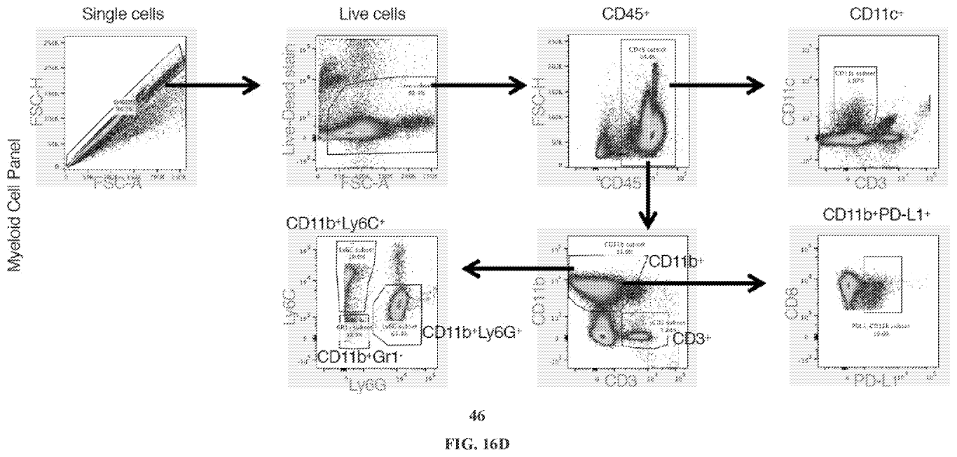

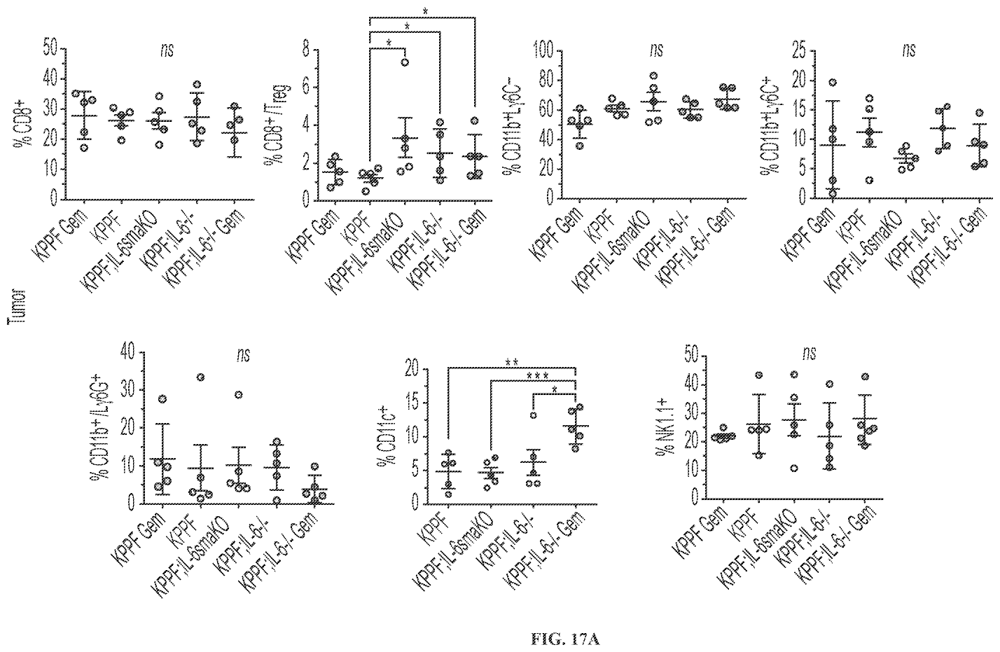

For the characterization of immune infiltration, tumors (from 2.5-month-old mice, treated with saline or gemcitabine for 2 weeks) were weighed, minced with gentleMACS Dissociator, and digested in 2 mL solution containing 1 mg/mL Liberase TL (Roche) and 0.2 mg/mL DNase I in DMEM media at 37° C. for 30 min. Spleens were weighed and filtered through a 100 μm mesh. Peripheral blood was collected with EDTA-tube, incubated with ACK lysis buffer for 5 min, and proceeded to mesh filtration. The tissue lysates were filtered through a 100 μm mesh before immunostaining. The subsequent single-cells suspension was stained with Fixable Viability Dye eFluor 780 (eBioscience) and antibodies specified in Table 2. The percentage positive cells were analyzed by FlowJo 10.1 and gated on CD45 positivity. Unstained, viability stain only, and single-stained beads (eBioscience) were used as compensation controls. Singlets were gated using forward scatter (FSC) height (FSC-H) and FSC area (FSC-A) event characteristics. The gating strategy is shown in FIGS. 16A-D.

Histopathological Scoring. Mouse tissues were fixed in 10% neutral buffered formalin, embedded in paraffin, and sectioned at 5 μm thickness. Sections were processed for haematoxylin and eosin (H&E) staining and/or Masson's trichrome staining (MTS) using Gomori's Trichome Stain Kit (38016SS2, Leica Biosystems). Histopathological assessments were conducted in a blinded fashion by scoring H&E-stained sections for relative percentages of the listed histopathological phenotypes. Tumor scores for orthotopic tumors were attributed on a scale from 1 (minor involvement) to 4 (extensive involvement), which evaluated on H&E sections of the entire pancreas the relative tumor involvement. Microscopic metastases were examined in H&E-stained tissue sections of the liver and lung. Positivity (one or more lesions in one tissue) was confirmed by CK19 staining. Images were obtained with a Leica DM 1000 LED microscope and an MC120 HD Microscope Camera with Las V4.4 Software (Leica).

scRNA Sequencing. The tumor of a PKTY;αSMA-RFP mouse was processed to obtain single cell suspension (see flow cytometry method section). Single cell Gel Bead-In-Emulsions (GEMs) generation and barcoding, post GEM-RT cleanup, and cDNA amplification, library construction, and Illumina-ready sequencing library generation were prepared by following the manufacturer's guidelines. High Sensitivity dsDNA Qubit kit was used to estimate the cDNA and library concentration. HS DNA Bioanalyzer was used for the quantification of cDNA. DNA 1000 Bioanalyzer was used for the quantification of libraries. Single-cell RNA-Seq data was processed by the Sequencing and Microarray Facility at MD Anderson Cancer Center. The “cloupe” files were generated by using Cell Ranger software pipelines following the manufacturer's guidelines. Further data analysis was performed by using 10× Genomics' Loupe Cell Browser software. 1118 cells from unfractionated tumor, 961 cells from αSMA-RFP sorted sample, and 340 from FAP-APC sorted sample were encapsulated using 10× Genomics' Chromium controller and Single Cell 3′ Reagent Kits v2 at the Sequencing and Microarray Facility at MD Anderson Cancer Center. Following capture and lysis, cDNA was synthesized and amplified to construct Illumina sequencing libraries. The libraries from about 1,000 cells per sample were sequenced with Illumina Nextseq 500 method at the Sequencing and Microarray Facility at MD Anderson Cancer Center. The run format was 26 cycles for read 1, 8 cycles index 1, and 124 cycles for read 2. The Fraction Reads in Cells scores ranged from 83.6 to 93.2%. The median genes per cell detected ranged from 872 up to 2870 genes per cell. The other QC metrics (% mapping of the sample, reads/cell, QC30 in RNA read) scores were all >65%. scRNA sequencing data was processed by the Sequencing and Microarray Facility at MD Anderson Cancer Center. Further data analysis was performed by using 10× Genomics' Loupe Cell Browser software. The genes identified using Loupe Cell Browser software as the top 100 differentially regulated genes in αSMA⁺ or FAP⁺ clusters were mapped onto ingenuity pathway analysis (IPA) networks. Human ‘normal’ pancreas was adjacent to pancreas tumor by at least 1.5 cm and was not matched to PDAC1 or PDAC2 (from a distinct patient). PDAC1 sample received 8 cycles of gemcitabine/abraxane. PDAC2 sample was treated with folfirinox. For the human tumor and normal adjacent samples, 214 cells from normal human pancreas, 562 cells from human PDAC1, 218 cells from human PDAC 2A, and 110 cells from PDAC 2B were encapsulated using 10× Genomics' Chromium controller and Single Cell 3′ Reagent Kits v2 at the Sequencing and Microarray Facility at MD Anderson Cancer Center. The libraries with maximal 5,000 cells per run were sequenced with Illumina Nextseq 500 method at the Sequencing and Microarray Facility at MD Anderson Cancer Center. The Fraction Reads in Cells scores ranged from 66% to 92.4%. The median genes per cell detected were 45 for normal human pancreas, 2,547 for human PDAC1, 506 for human PDAC2A, and 542 for human PDAC2B. The percent mapping of the transcriptome ranged from 34.7% up to 58.5%. The QC30 in RNA read scores were all >65%. Further data analysis was performed by using 10× Genomics' Loupe Cell Browser software.

Isolation of primary pancreatic adenocarcinoma cells and myofibroblasts from PDAC tissues. Establishment of primary PDAC cell and myofibroblast lines was conducted as previously depicted with minor modifications (Zheng et al., 2015). Fresh PDAC tissues from KPPF;IL-6^{smaKO/WT};R26^{Dual} mice were minced with sterile lancets, digested with collagenase IV (17104019, Gibco, 4 mg/mL)/dispase II (17105041, Gibco, 4 mg/mL)/DMEM at 37° C. for 1 h, filtered by 70 μm cell strainers, resuspended in DMEM/20% FBS, and then seeded on type-I collagen-coated dishes (354401, Corning). Cells were cultured in DMEM medium containing 20% FBS and 1% penicillin/streptomycin/amphotericin B (PSA) antibiotic mixture. Pdx1-expressing cancer cells and αSMA-expressing myofibroblasts were further purified by FACS (BD FACSAria™ II sorter) based on EGFP and tdTomato signals, respectively. The sorted cells were subsequently maintained in vitro. All studies were performed on cells cultivated less than 25 passages. DNA from these primary cell lines was extracted using the DNA Mini Kit (51304 QIAGEN).

Gene expression analyses and global gene expression profiling. RNA was extracted using the RNeasy Mini Kit as directed (74104, QIAGEN) from PDAC tumor tissues of KPPF, KPPF;IL-6^{smaKO}, and KPPF;IL-6^{-/-} mice. cDNA was synthetized using High Capacity cDNA Reverse Transcription Kit (4368814, Applied Biosystems). The expression level of detected genes was normalized to that of Gapdh housekeeping gene. The relative expression data is presented as fold change (2^{ΔΔCt}) with the control group normalized to a fold value of 1. Statistical analyses were performed on ΔCt. Primer sequences for detected genes are listed below. Mouse genes and primers are as follows: GAPDH F 5′-AGGTCGGTGTGAACGGATTTG-3′ (SEQ ID NO: 1); GAPDH R 5′-TGTAGACCATGTAGTTGAGGTCA-3′ (SEQ ID NO: 2); IL-6 F 5′-GCTTAATTACACATGTTCTCTGGGAAA-3′ (SEQ ID NO: 3); IL-6 R 5′-CAAGTGCATCATCGTTGTTCATAC-3′ (SEQ ID NO: 4); IL-1β F 5′-GGGCTGCTTCCAAACCTTTG-3′ (SEQ ID NO: 5); IL-1β R 5′-TGATACTGCCTGCCTGAAGCTC-3′ (SEQ ID NO: 6); αSMA (Acta2) F 5′-GTCCCAGACATCAGGGAGTAA-3′ (SEQ ID NO: 7); αSMA R 5′-TCGGATACTTCAGCGTCAGGA-3′ (SEQ ID NO: 8).

Total RNA was also isolated from tumors of age-matched PKT;αSMA-TK, and PKT;FAP-TK mice (n=3 mice per in each group), that were administrated with GCV or PBS. RNA extraction was carried out using the QIAGEN RNeasy Mini Kit and submitted to the Microarray Core Facility at MD Anderson Cancer Center. Gene expression analysis was performed using Affymetrix MTA 1.0 Genechip. The Limma package (Smyth, 2005) from R Bioconductor was used for quantile normalization of expression arrays and to analyze differential gene expression between the TK groups (PKT;αSMA-TK and the PKT-FAP-TK groups) and their respective control (PKT-αSMA-TK^{control} and PKT;FAP-TK^{control}) groups (p≤0.05 and fold change ≥1.2). Analyses of differentially expressed pathways between the TK and control groups were performed using Gene Set Enrichment Analysis (GSEA) (Subramanian et al., 2005). Gene expression microarray data was deposited in GEO, which is available on the world wide web at ncbi.nlm.nih.gov/geo/query/acc.cgi?acc=GSE120577.

TCGA Data Analysis. The mRNA expression profiles of 179 pancreatic adenocarcinoma cases from the Cancer Genome Atlas (TCGA) database were analyzed after downloading the related mRNA expression data (RNA Seq V2 RSEM) with the cBioPortal for Cancer Genomics (available on the world wide web at cbioportal.org/) (Cerami et al., 2012). All cases were divided into two (IL-6-High and IL-6-Low) groups according to their relative IL-6 mRNA level (normalized to the ACTB housekeeping gene).

Statistical Analyses. The statistical tests used for the comparative analyses presented are listed in the figure legends. Statistical analyses were carried out using GraphPad Prism (GraphPad Software). Kaplan-Meier plots were used for survival analysis and the log rank Mantel-Cox test was used to evaluate statistical differences with GraphPad Prism. Error bars represent standard error of the mean (sem) unless specified in the figure legends. Statistical significance was defined as P<0.05.

### Example 1—PDAC Fibroblastic Stroma is Heterogeneous

To define the fibroblast subsets associated with the desmoplastic reaction of PDAC lesions, the spontaneously arising pancreatic tumors of Ptf1a^{cre/+};LSL-Kras^{G12D/+}; Tgfbr2^{loxP/loxP} (PKT) genetically engineered mice (GEMs, Table 1; n=11) were immunolabeled for prototypical mesenchymal gene products (LeBleu & Kalluri, 2018). These included alpha-smooth muscle actin (Acat2, αSMA), platelet growth factor receptor alpha (Pdgfra, PDGFRα), fibroblast specific protein 1 (S100A4, Fsp1), and vimentin (Vim, referred also therein as Vim). Fibroblast activation protein (FAP) identification could not be included because of poor labeling by the anti-FAP antibodies in the multiplex staining procedure. Single stained controls were analyzed using the same spectral unmixing algorithm employed on the multiplex sections and showed no overlap between the different fluorophores. Together with DAPI nuclear staining and cytokeratin 8 (CK8) epithelial immunolabeling, the quantitative multiplex immunofluorescence analysis revealed that the chosen mesenchymal markers captured approximately 36.27% of all cells in the tumors, with the remaining cells being cancer cells (CK8⁺; 53.59%) or unlabeled cells (10.14%). The unlabeled cells likely comprise vascular and immune cells that are not captured in the mesenchymal staining panel. Although cancer cells were noted to express fibroblast markers (possibly labeling cells with an epithelial to mesenchymal transition (EMT) program), the subsequent analyses was restricted to the mesenchymal stroma by excluding cells that expressed the epithelial marker (CK8). Analysis of the relative mesenchymal marker overlap to define distinct mesenchymal cell subtypes revealed that the dominant mesenchymal cell population in these tumors was αSMA⁺ cells (30.40%), followed by PDGFRα⁺ cells (FIG. 1A). Up to 69% of the mesenchymal stroma was comprised of cells expressing either αSMA, PDGFRα, or both, and devoid of vimentin and Fsp1 (FIG. 1A). Expression of PDGFRα overlapped with other mesenchymal markers (αSMA, vimentin, or Fsp1), and vimentin expression was the most promiscuous of markers, showing co-positivity with all other mesenchymal markers evaluated (FIG. 1A). This analysis illustrated that fibroblast identity in PDAC, characterized by a defined set of markers, identified a highly heterogeneous fibroblast composition, with fibroblasts specifically labeled by αSMA as the dominant mesenchymal species. Notably, using immunolabeling of PDAC tissue sections, minimal overlap in αSMA and FAP was observed in murine (Ozdemir et al., 2014) and human PDAC stroma (FIG. 1B). Further, PKT mice were engineered with lineage tracing of cancer cells (LSL-YFP) and αSMA⁺ CAFs (αSMA-RFP transgene), which enabled the flow cytometry capture of cancer cells (YPF) and αSMA⁺ CAFs (YFP⁻, RFP⁺). Flow cytometry analyses of these tumors for endogenous YFP and RFP (αSMA) signal, together with FAP immunolabeling (APC labeled), also revealed minimal overlap between the αSMA and FAP expression in stromal cells (FIG. 1C).

### Example 2—scRNA-Seq Analyses Identify αSMA⁺ and FAP⁺ as Distinct Subsets of CAFs in PDAC

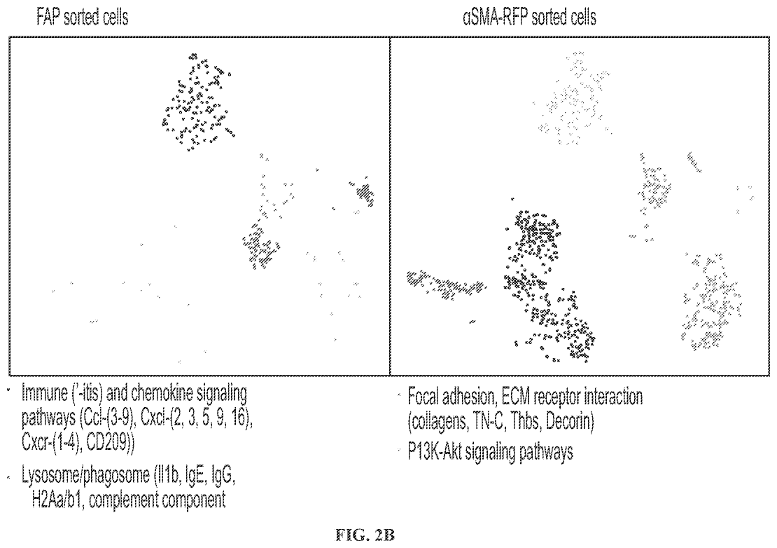

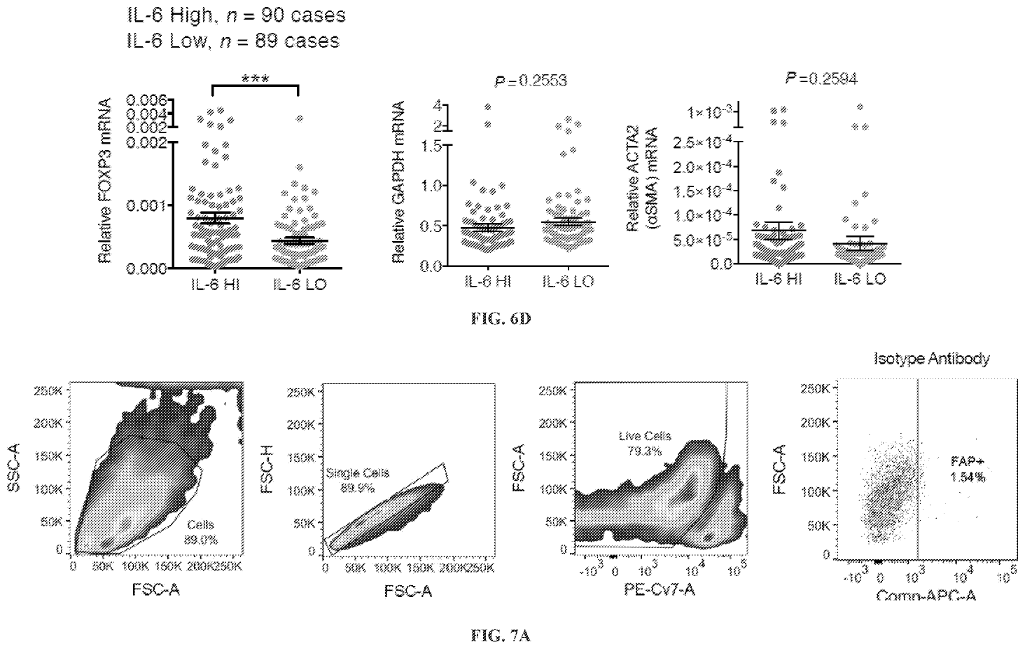

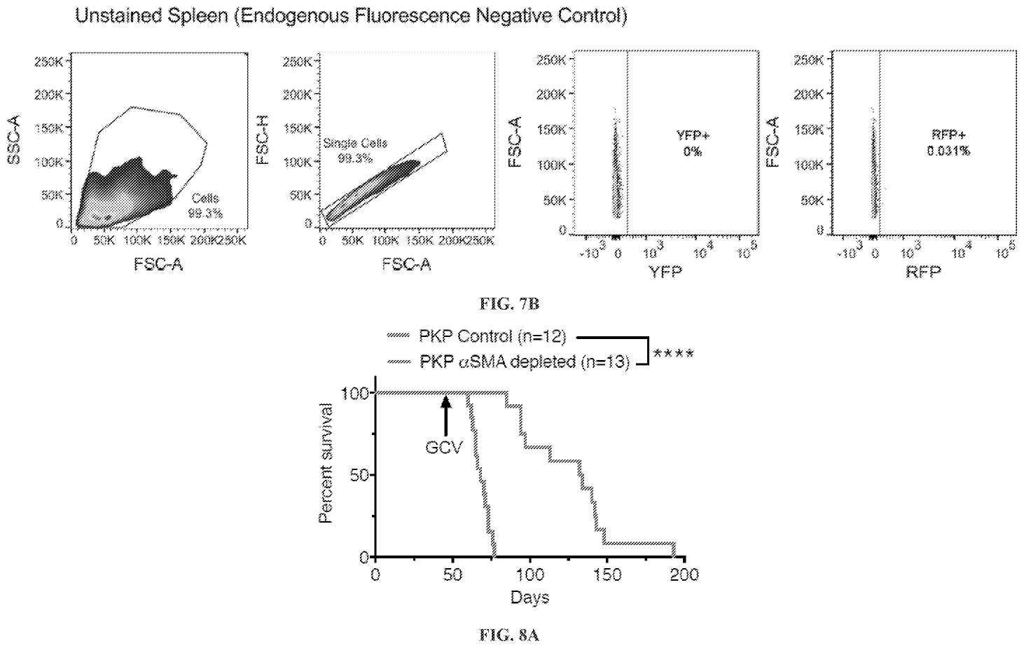

To further resolve the functional heterogeneity of fibroblasts in PDAC, single cell RNA sequencing (scRNA-Seq) of unfractionated cells from PKT tumors was performed. Dimensional reduction and representation of specific gene expression profile in t-SNE plots revealed that single cell isolation predominantly yielded immune cells (myeloid (Itgam; Itgb2; S100a8; Ccl3; Apoe; Itgax; Adgre1) and lymphoid (Ptprc; Cd3d; Cd4; Gzmb; Cd8a; Cd19; CD69; CD79a; MS4a2; Cd3e; Cd79b) lineages), with smaller clusters (representations) of epithelial cells (Krt19; Muc1; Krt18; Muc5ac) and fibroblasts (Vim; Acta2; Fap; Thy1; Col3a1; S100a4; C3; Des; Cxcl12; Pecam1; Pdgfra; Pdgfrb). To enrich for the αSMA⁺ and FAP⁺ fibroblasts, flow cytometry was performed with purified αSMA-RFP⁺ and FAP (immunolabeled) fibroblasts prior to scRNA-Seq (FIG. 1D, FIGS. 7A-B). Distinct clusters of cells emerged from αSMA⁺ and FAP⁺ enriched populations respectively, with one observed common cluster between the two (cluster 3) (FIG. 2A, left panel). The αSMA⁺ enriched clusters (cluster 1, 2, 4, and 6) included cancer cells (CK19, CD18, Muc1) undergoing epithelial to mesenchymal transition (EMT, cluster 2) and other stromal cells (clusters 1, 4, and 6) (FIG. 2A). Among the αSMA⁺ enriched clusters, a cluster of cells with a T-lymphocyte gene signature (CD3, CD4, CD8; cluster 4), a cluster of cells with a macrophage gene signature (CCLS, CCL22; cluster 6), and a cluster of cells with a collagen/extracellular matrix (ECM) gene signature (Col1α1, MMP, Lox; cluster 1) were observed (FIG. 2A). In contrast, among the FAP⁺ enriched clusters, a cluster of cells with a B-lymphocyte gene signature (CD79a/b, CD19; cluster 5), and a cluster of cells with a neutrophil-like gene signature (NGP, Retnlg; cluster 7), were observed (FIG. 2A). The cluster shared by the αSMA⁺ and FAP⁺ sorted cells presented with a myeloid gene expression signature (F4/80, Mac-1, CD11c; FIG. 2A). Clusters 8 and 9, depicting broken cells (mtRNA) or red blood cell contaminant (Hba/Hbb), were minor clusters and disregarded in the subsequent analyses (FIG. 2A). The T- and B-lymphocyte gene signatures observed in defined clusters of αSMA⁺ and FAP⁺ sorted CAFs, respectively, were indicative of gene expression associated with immune cells in CAFs. Its non-random pattern among the cell clusters argues against a potential immune cell contaminant from the flow cytometry-based enrichment of αSMA⁺ and FAP⁺ cells. Further, there were only few cancer cells with an EMT program gene signature captured in the FAP⁺ sorted cells.

Next, αSMA⁺ and FAP⁺ CAFs (excluding the cancer cells captured in the αSMA⁺ sorted cells) were defined and the genes enriched in these subpopulations were ascertained (FIG. 2B). αSMA⁺ CAFs were enriched in transcripts associated with focal adhesion, ECM receptor interaction, and PI3K-Akt signaling, whereas FAP⁺ mesenchymal cells were enriched in transcripts associated with immune and chemokine signaling, and lysosome and phagosome activity (FIG. 2B). Exemplary genes that were upregulated in αSMA and downregulated in FAP include Col3a1, Dcn, Serpinh1, Col1a1, Crlf1, Mgp, Acta2, Fstl1, Myl9, Ctgf, Igfbp5, Sparc, Bgn, Serpinf1, Igfbp7, Cpxm1, Tnc, Col1a2, Loxl1, Rbp1, Sparcl1, Postn, Col5a2, Col6a1, Mfap2, Lum, CCl11, Aebp1, Rarres2, Gm13889, Mylk, Ndufa412, Oaf, Gpx8, Mfap4, Ccdc80, Mmp2, Serping1, Cyr61, Mfap5, Col4a2, Fxyd6, Sfrp1, Rasl11a, Mdk, Cald1, Serpine2, Lox, Snhg18, Cygb, Tagln, Penk, Cdhl1, Col8a1, Ppic, Rgs5, Tpm2, Rxyd1, Itm2a, and Prkcdbp. Exemplary genes that were downregulated in αSMA and upregulated in FAP include S100a9, S100a8, Jchain, Ccl3, Ccl6, Wfdc17, Il1b, Rac2, Ctss, Cd52, Retnig, Spi1, Lcp1, C1qc, Ccl4, Tyrobp, Clec4n, Fcgr2b, C1qa, Bcl2a1b, Ms4a6c, Laptm5, C1qb, Plek, Bcl2a1d, Coro1a, Lyz2, H2-Aa, Pf4, Fcer1g, Ptpn18, Ccl8, Arg1, Rgs1, Ccl9, Ccl17, Ccl14, H2-Ab1, Cxcr4, Cd74, Srgn, Apoe, Csf1r, AA467197, Alox5ap, Fcgr3, Cd53, Ccrl2, Acp5, Cxcl2, Ucp2, Rgs10, Tpd52, Lgals3, Tgfbi, Fam49b, Trem2, Lst1, Mafb, and Msrb1. These data further highlight that transcriptomes associated with αSMA⁺ and FAP⁺ CAFs are distinct and suggest a role for αSMA⁺ CAFs in extracellular matrix remodeling and FAP⁺ CAFs in the regulation of the tumor immune response.

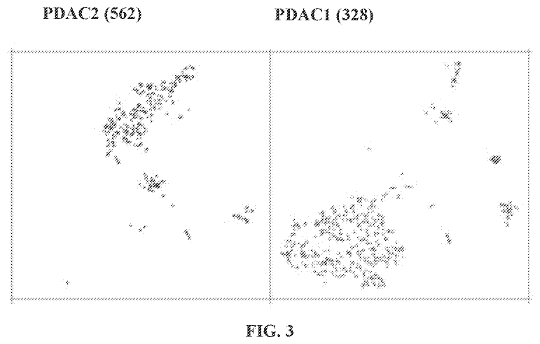

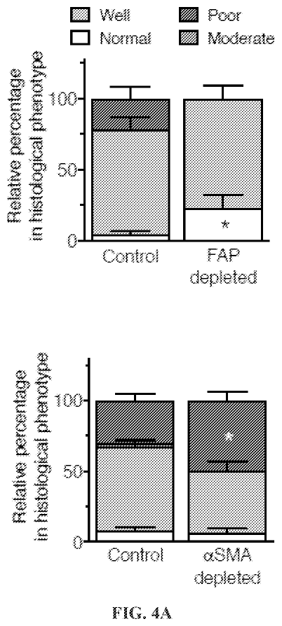

The scRNA-Seq analyses of human PDAC tumors also revealed an enrichment in immune and epithelial clusters, with heterogeneity in the captures of these clusters from patient to patient (FIG. 3). Transcriptomic analyses of single cells from PDAC tumors obtained from two different patients (PDAC 1 and PDAC 2) revealed similar clusters in technical replicates (PDAC 2A and PDAC 2B); however, the PDAC 1 cluster consisted predominantly of immune cells (PTPRC, CD69), whereas PDAC 2 clusters were enriched in epithelial cells (CK19, MUC1, CK18). When viable cells were captured in the scRNA-Seq analyses of healthy human pancreas, αSMA⁺ mesenchymal cells could still be detected, but their frequency of detection was low.

### Example 3—αSMA⁺ and FAP⁺ CAFs Exhibit Opposing Functions in PDAC Progression

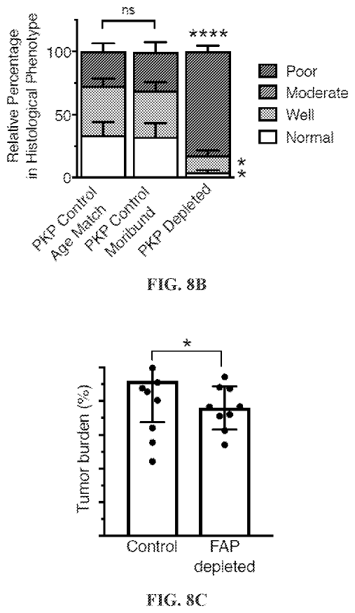

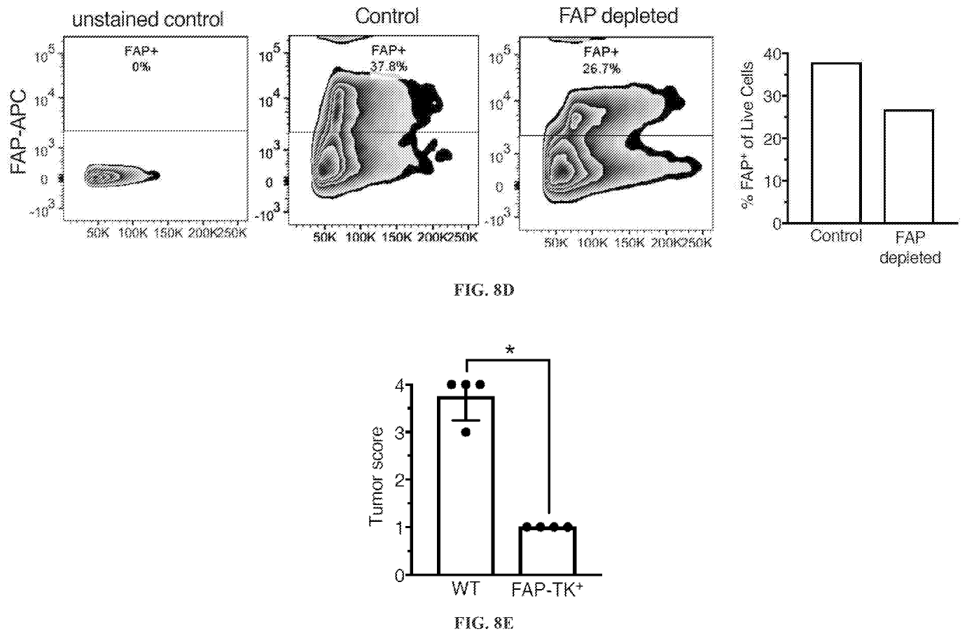

A genetic approach for targeting proliferating αSMA⁺ mesenchymal cells in PDAC GEMs, wherein depletion of αSMA⁺ CAFs accelerated PDAC progression, has been previously reported (Ozdemir et al., 2014). These findings and others also highlighted the potential anti-tumor function of PDAC CAFs (Ozdemir et al., 2014; Feig et al., 2013; Kraman et al., 2010; Rhim et al., 2014). In light of such reports and guided by the distinct transcriptomic profiles of αSMA⁺ and FAP⁺ CAFs, PKT GEMs were generated with the ability to specifically deplete αSMA⁺ CAFs (αSMA-TK), as well as PKT GEMs with the ability to specifically deplete FAP-TK⁺ CAFs. Similar to the αSMA⁺ cells, the FAP-TK transgene enabled specific depletion of proliferating FAP-expressing cells upon ganciclovir administration. Depletion of αSMA⁺ CAFs resulted in a more aggressive PDAC phenotype (FIGS. 4A-B). A similar phenotype, together with decreased survival, is also observed when αSMA⁺ CAFs are depleted in the PKP GEM (Ptf1a^{cre/+};LSL-Kras^{G12D/+};Trp53^{R172H/+}) (FIGS. 8A-B). In contrast, the depletion of FAP⁺ CAFs resulted in suppression of PDAC phenotype (FIGS. 4A-B, FIGS. 8C-D). Lower tumor burden was also observed when FAP⁺ CAFs were depleted in the context of orthotopically implanted PDAC tumors, when compared to control mice (FIG. 8E).

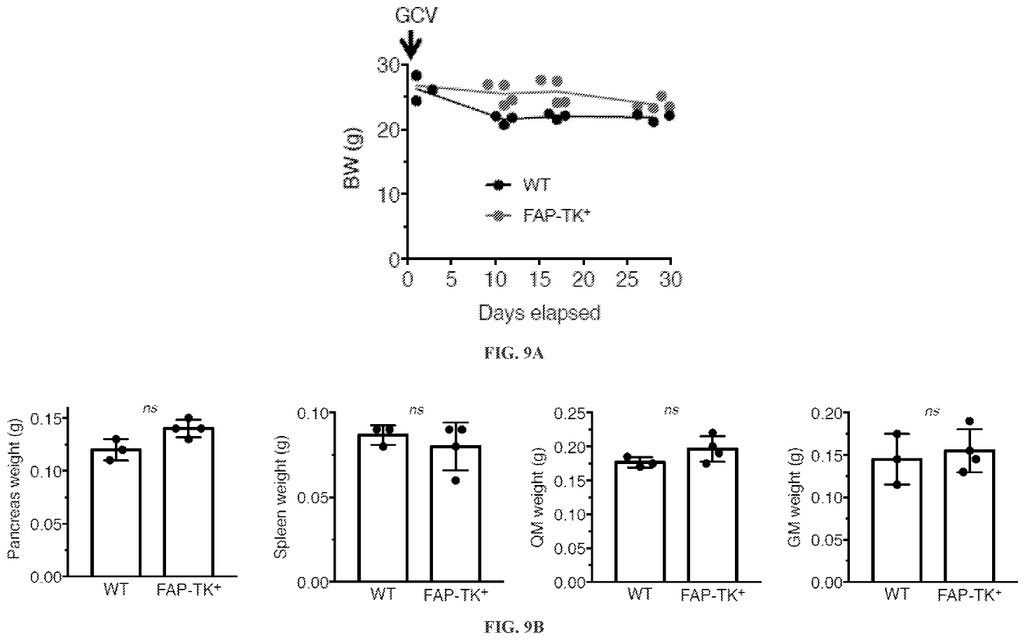

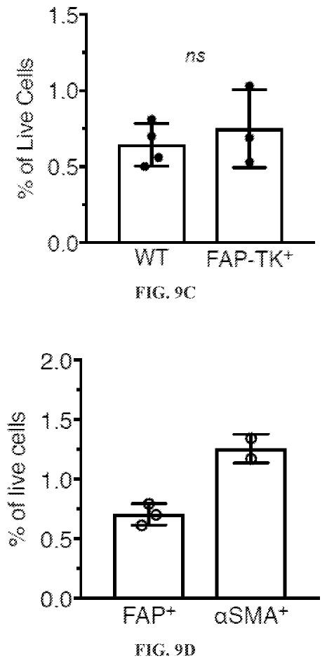

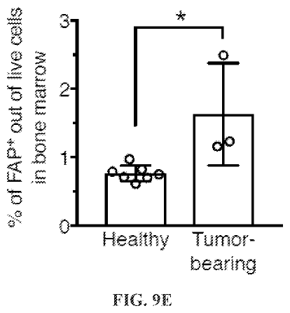

Previous studies implicated FAP targeting in the development of a cachexic phenotype in mice (Roberts et al., 2013; Tran et al., 2013). The genetic targeting strategy, limited to actively proliferating cells, was not associated with body weight loss over time, nor muscle wasting (FIGS. 9A-B). No loss in FAP⁺ cells in the spleen of healthy mice was noted with the genetic strategy (FIG. 9C). In light of the scRNA-Seq results obtained from tumor-derived αSMA⁺ and FAP⁺ CAFs, indicative of lymphocyte and myeloid gene signatures (FIG. 2A), the frequency of αSMA⁺ and FAP⁺ cells in the bone marrow of healthy mice was ascertained. αSMA⁺ and FAP⁺ cells were minor cell populations in the unfractionated femoral and tibia bone marrow flush (less than 1.5%) (FIG. 9D). Interestingly, FAP⁺ cell frequency in the bone marrow was elevated in PDAC bearing mice, when compared to healthy control mice, raising the bone marrow as a possible source of FAP⁺ CAFs in PDAC tumors (FIG. 9E).

### Example 4—Distinct Impact of αSMA⁺ and FAP⁺ CAFs Depletion on PDAC Transcriptome

To decipher the mechanistic underpinning for the opposing functions of αSMA⁺ and FAP⁺ CAFs in PDAC progression, global transcriptomic analyses of control, αSMA⁺ cells-depleted tumors, and FAP⁺ cells-depleted tumors was performed. Comparative analyses of common and non-overlapping genes following the depletion of αSMA⁺ or FAP⁺ CAFs revealed minimal overlap, for both downregulated and upregulated genes (FIG. 4C). To ascertain whether such distinct transcriptomic profiles were associated with specific biological processes, the overlap in pathways defined by changes in transcript levels for defined gene sets was evaluated. In αSMA⁺ CAFs-depleted tumors, gene expression changes were largely associated with upregulation of pathways associated with ribosomal, lysosomal, and phagocytic activity, chemokine signaling, VEGF signaling, and B and T cell receptor signaling (717 upregulated pathways, FIG. 4D). Interestingly, these pathways were downregulated in FAP⁺ CAFs-depleted tumors, underscoring the opposite functions of αSMA⁺ and FAP⁺ CAFs in PDAC (98 downregulated pathways, FIG. 4D). Although this evaluation cannot distinguish transcriptomic changes associated with specific cell types in the tumors, it is noted that when FAP⁺ CAFs are depleted, lysosomal and phagocytic activity and chemokine signaling pathways were downregulated in the tumor transcriptome (FIG. 4D). The same pathways were also found enriched in the scRNA-Seq analyses of FAP⁺ CAFs enriched from PDAC tumors (FIG. 2B). These results suggest that the downregulated pathways in FAP⁺ CAFs-depleted tumors reflect changes associated with FAP⁺ cells observed by scRNA-Seq analysis. Upregulated pathways in FAP⁺ CAFs-depleted tumors included cytoplasmic trafficking (ARF6), cell adhesion, and ECM degradation (FIG. 4D). Interestingly, scRNA-Seq analyses of αSMA⁺ cells enriched from PDAC tumors indicated they likely engage in ECM-cell surface receptor interactions and cell adhesion (FIG. 2B). These data support the notion that the depletion of FAP⁺ CAFs yielding improved PDAC histopathology via attenuation of inflammation (FIG. 4A), and via upregulation of anti-tumor activity associated with αSMA⁺ CAFs (FIGS. 4A, 4C). Commonly upregulated pathways in tumors depleted of either αSMA⁺ CAFs or FAP⁺ CAFs include hypoxia-inducible factor-1 (HIF-1), P53, and apoptotic pathways, and there were no commonly downregulated pathways identified, supporting a distinct impact on the transcriptome of the tumors depleted of either αSMA⁺ CAFs or FAP⁺ CAFs (FIG. 4C).

### Example 5—Depletion of αSMA⁺ and FAP⁺ CAFs Remodels PDAC Tumor Stroma in a Distinct Manner

To define the impact of depletion of αSMA⁺ vs. FAP⁺ CAFs in PDAC, the relative changes in the frequencies of each CAF subset (defined in FIG. 1A) were measured in tumors. The total CAF population (as captured by this assay) was significantly reduced when αSMA⁺ CAFs were depleted (˜48% reduction in in CAFs examined), whereas, depletion of FAP⁺ CAFs marginally reduced the CAF population (˜11.2% reduction, FIG. 4E). In both the contexts of αSMA⁺ or FAP⁺ CAF depletion, the composition of CAF subsets was significantly altered. These changes were more pronounced when αSMA⁺ CAFs were depleted in contrast to when FAP⁺ CAFs were depleted (FIG. 4E), underscoring their distinct impact on the PDAC tumor microenvironment and progression. Out of all αSMA⁺ CAFs, αSMA⁺ CAFs depletion predominantly reduced the αSMA⁺ PDGFRα⁺ subset, whereas FAP⁺ CAFs depletion enhanced αSMA⁺ CAFs but preserved the frequencies of most other subsets of CAFs co-expressing αSMA (FIG. 4E). Further, αSMA⁺ CAFs depletion also resulted in an increase in the frequency of FSP1⁺ cells (FSP1⁺ cells and FSP1⁺ and αSMA⁺ cells), whereas FAP⁺ CAFs depletion was associated with an increase in Vim⁺ CAFs but overall reduction in PDGFRα⁺ CAFs frequency (PDGFRα⁺, PDGFRα⁺ Vim⁺, and PDGFRα⁺ αSMA⁺) (FIG. 4E). Depletion of FAP⁺ CAFs resulted in maintenance of the frequency of αSMA⁺ CAFs, thereby possibly preserving their tumor restraining properties (FIG. 4A). The scRNA-Seq of αSMA⁺ and FAP⁺ CAFs indicated a complex mesenchymal gene expression overlap in the αSMA⁺ cluster, compared with the FAP⁺ cluster. These results suggest that the αSMA⁺ cluster captures a more heterogeneous and complex population of CAFs, and as such, FAP⁺ CAFs may constitute a more progenitor-like mesenchymal cell population. These findings (FIG. 4E), together with the genetic targeting studies and gene expression profiling of PDAC GEM (FIGS. 4A-D), support distinct functions of αSMA⁺ and FAP⁺ CAFs in PDAC, with αSMA⁺ CAFs displaying tumor suppressing (TS)-CAF functions and FAP⁺ CAFs displaying tumor promoting (TP)-CAF functions.

### Example 6—αSMA⁺ CAFs-Derived IL-6 Confers Resistance to Gemcitabine

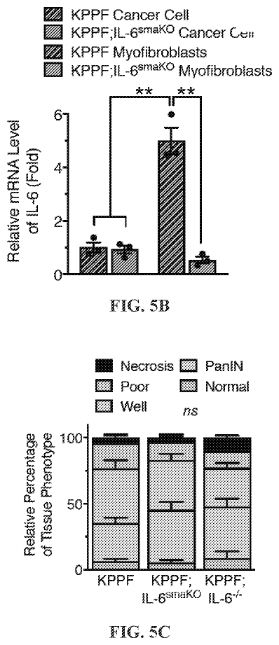

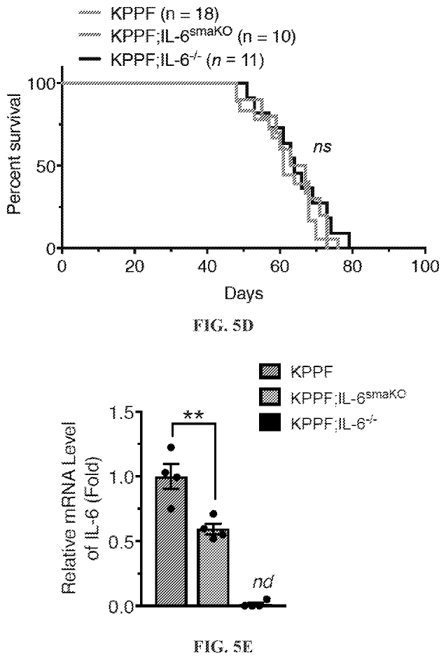

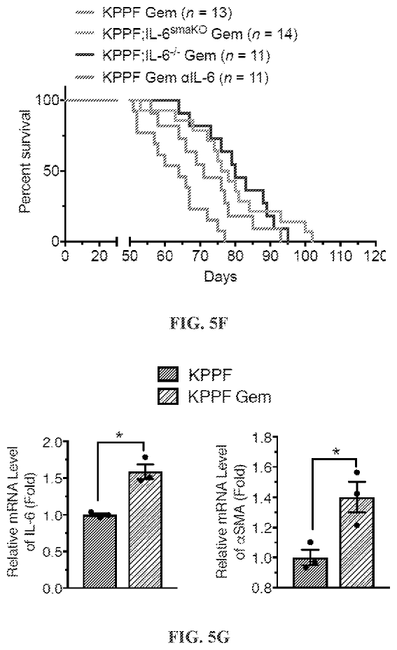

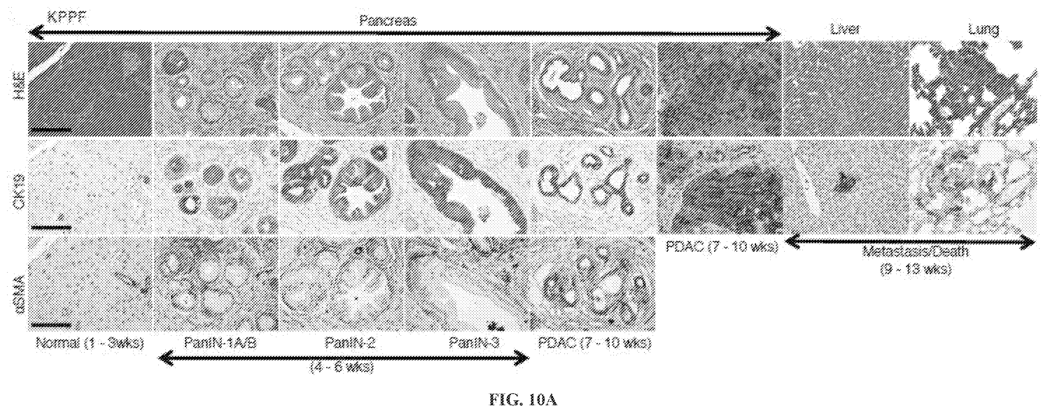

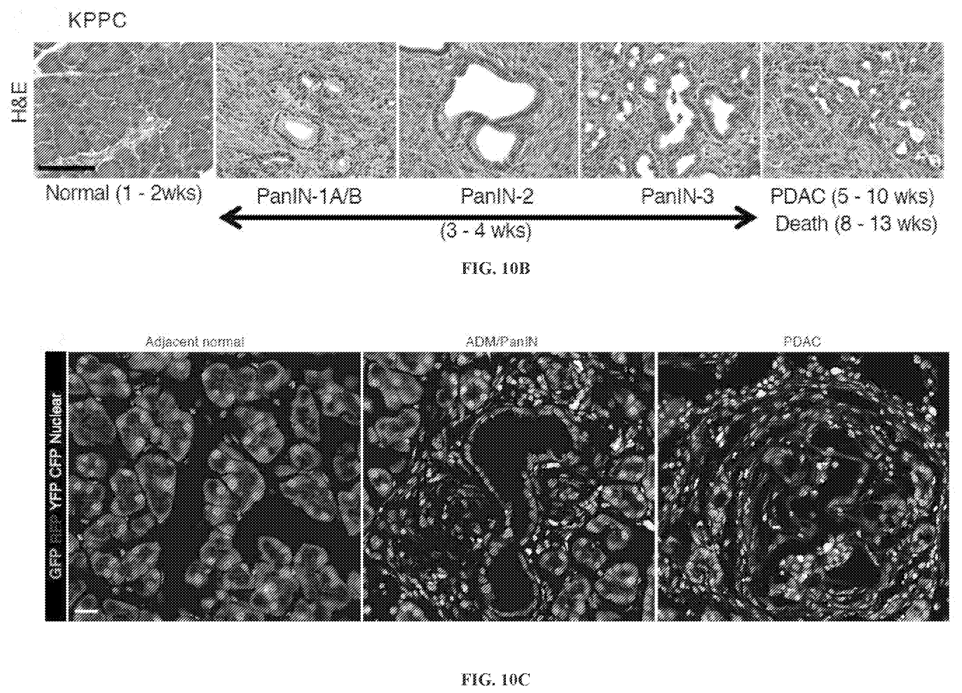

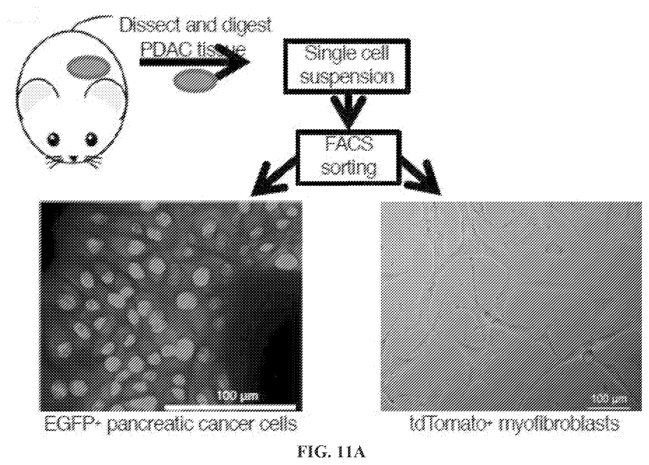

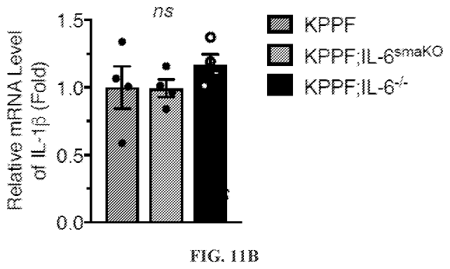

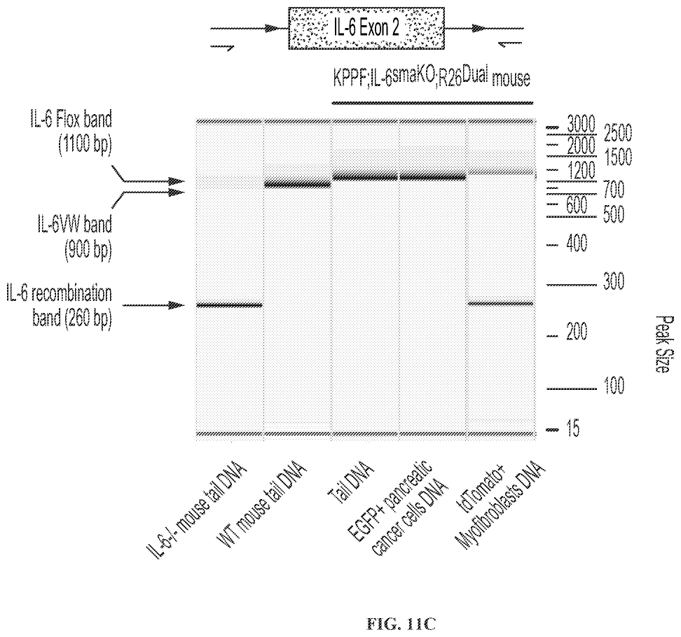

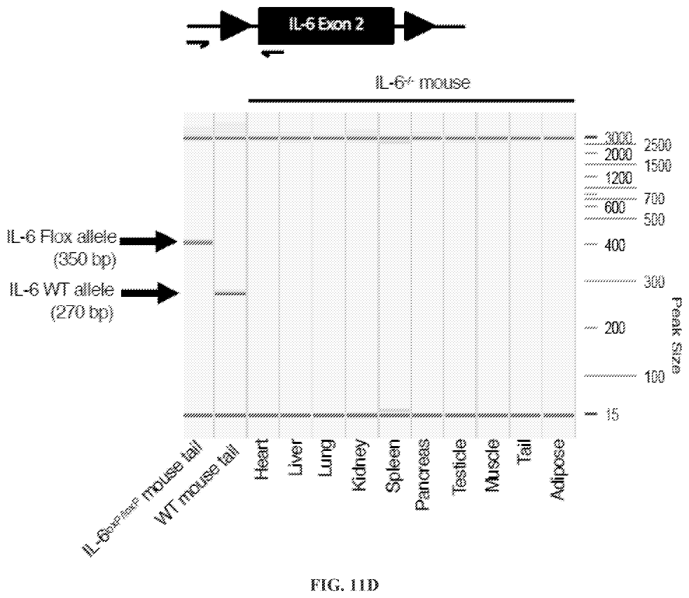

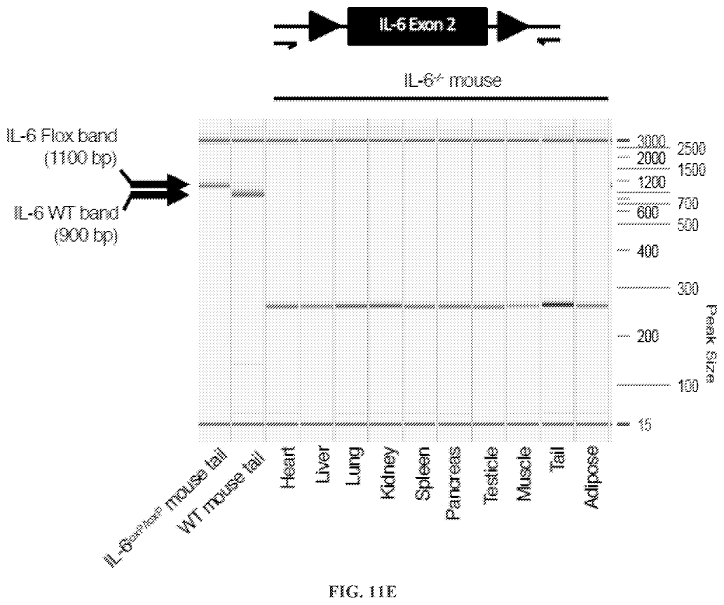

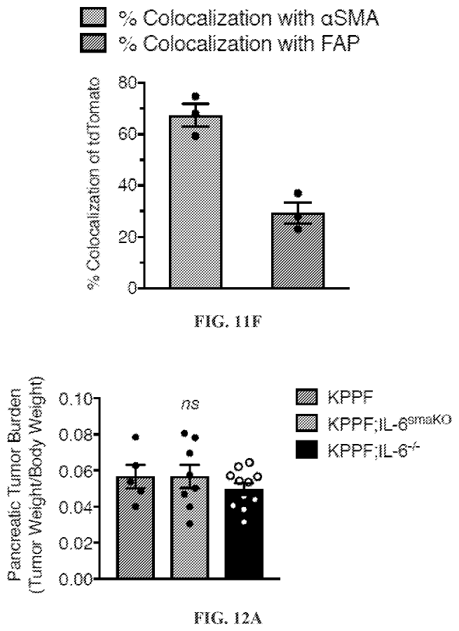

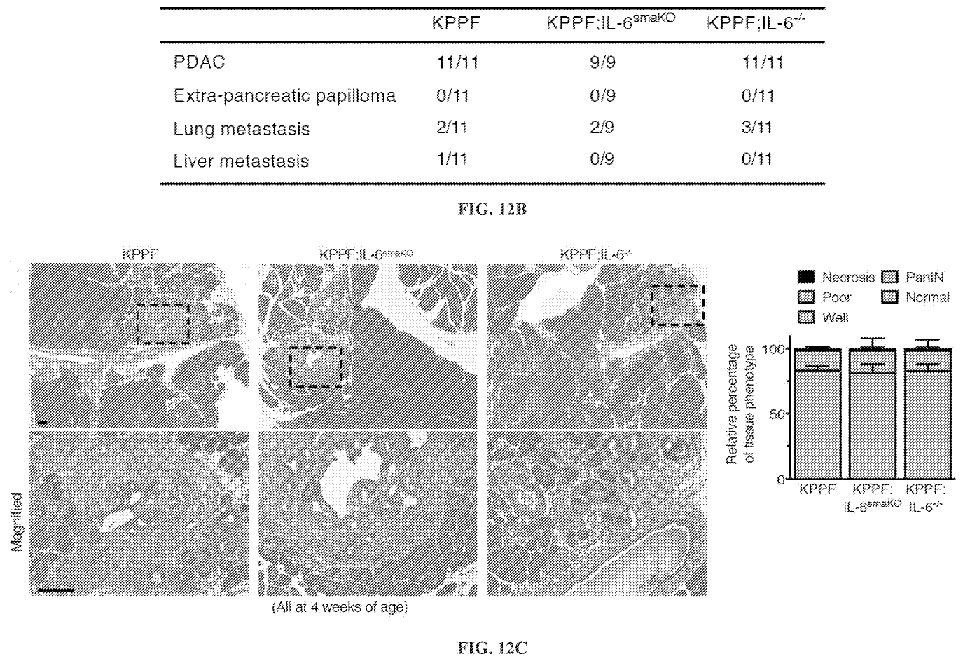

scRNA-Seq analyses of αSMA⁺ CAFs or FAP⁺ CAFs from PDAC tumors underscored their heterogeneous phenotype and present with distinct transcriptomic profiles reminiscent of several immune cell types, including myeloid and lymphocyte lineages (FIG. 2A). These data, together with the phenotypic and transcriptomic changes associated with αSMA⁺ CAFs or FAP⁺ CAFs depletion in PDAC tumors, support the notion that αSMA⁺ CAFs and FAP⁺ CAFs regulate tumor immunity in a distinct manner. In light of studies implicating the stromal IL-6 as a critical mediator of polarization of immune cells (Lesina et al., 2014), the scRNA-Seq data was queried to define CAF subpopulations enriched for IL-6 transcripts. The IL-6 transcript was predominantly associated with the αSMA⁺ CAFs-enriched cluster (46% of αSMA⁺ cells vs. 3% of FAP⁺ cells were positive for high IL-6 transcript levels, FIG. 5A). To investigate the functional contribution of αSMA⁺ CAFs-derived IL-6 to PDAC progression, GEMs were generated in which two distinct gene recombination systems (flippase- or Cre-mediated recombinase, Table 1) independently drive cancer formation (Pdx1-Flp;FSF-Kras^{G12D/+};TP53^{frt/frt}; KPPF) and conditional gene recombination in αSMA⁺ CAFs (αSMA-Cre; foxed-gene of interest) (Chen et al., 2018). KPPF mice presented with a similar disease progression as the comparable Cre-driven model (Pdx1-Cre;LSL-Kras^{G12D/+};TP53^{loxP/loxP}; KPPC, FIGS. 10A-B). Recombination in the αSMA⁺ CAFs of KPPF;αSMA-Cre;R26^{Confetti} reporter mice was visualized by capture of GFP, RFP, YFP, and CFP fluorescent cells in the desmoplastic reaction associated with PDAC (FIG. 10C). Using KPPF;αSMA-Cre;R26^{Dual} reporter mice (Table 1), IL-6 transcripts enrichment in purified tdTomato⁺ fibroblasts was confirmed, and higher IL-6 expression in αSMA⁺ CAFs compared to cancer cells was noted (FIG. 5B). Further, KPPF;αSMA-Cre;R26^{Dual} reporter mice were bred to a conditional IL-6 gene knockout allele (KPPF; IL-6^{smaKO}), effectively abrogating IL-6 transcription in αSMA⁺ CAFs (FIG. 5B). KPPF mice were also bred with systemic IL-6 knockout mice (KPPF; IL-6^{-/-}). Disease progression and PDAC histology was similar in both the conditional and systemic IL6 knock out mice when compared to the KPPF control (FIGS. 5C-D, FIGS. 12A-C) (Wormann et al., 2016). IL-6 levels were significantly decreased in tumors of KPPF;IL-6^{smaKO} mice compared to KPPF control tumors, and absent in KPPF;IL-6^{-/-} tumors (FIG. 5E). IL-1β transcripts levels were unchanged (control, FIG. 11A). Genomic recombination events captured by PCR reactions in tumors and control organs also confirmed the specificity of the genetic strategy employed to ensure conditional deletion of IL-6 in αSMA⁺ cells (FIGS. 11B-D). Interestingly, approximately ˜65% of the αSMA-Cre lineage-traced (tdTomato⁺) CAFs co-localized with αSMA immunofluorescent staining (FIG. 11E).

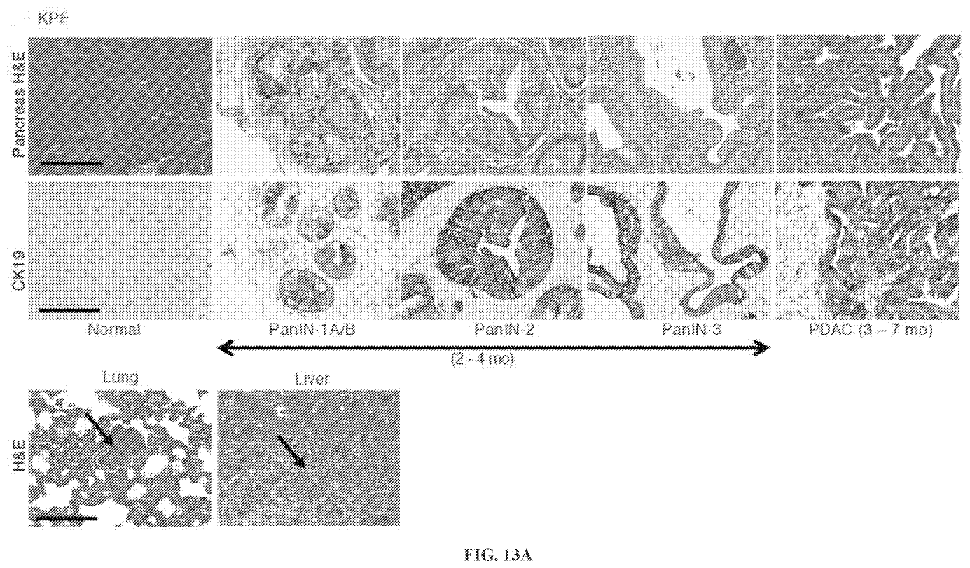

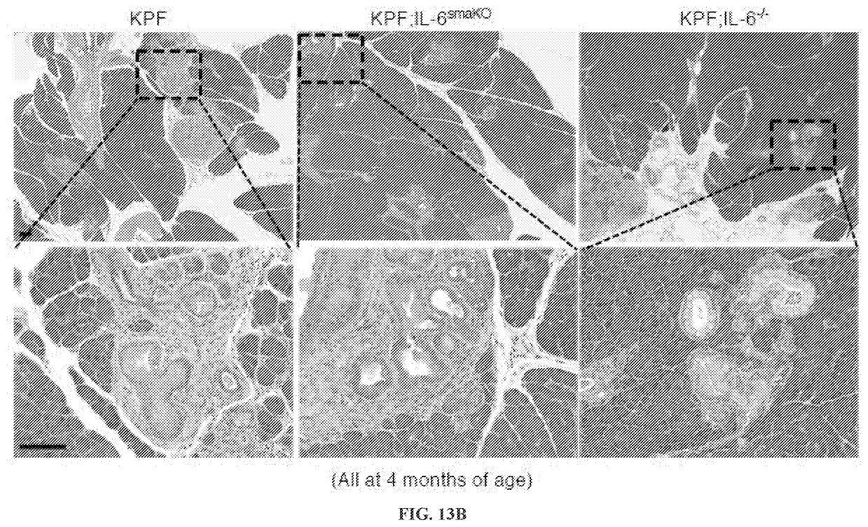

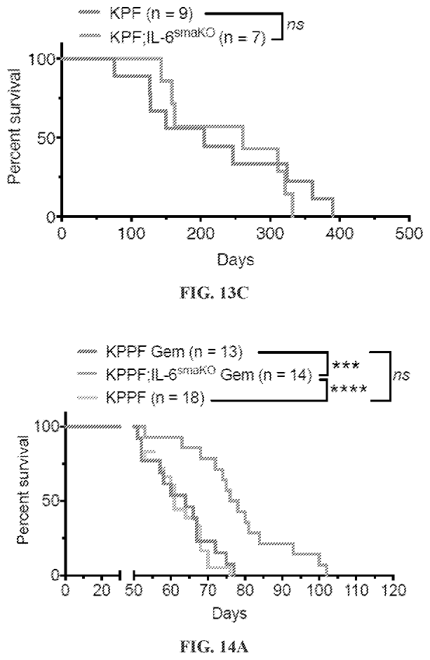

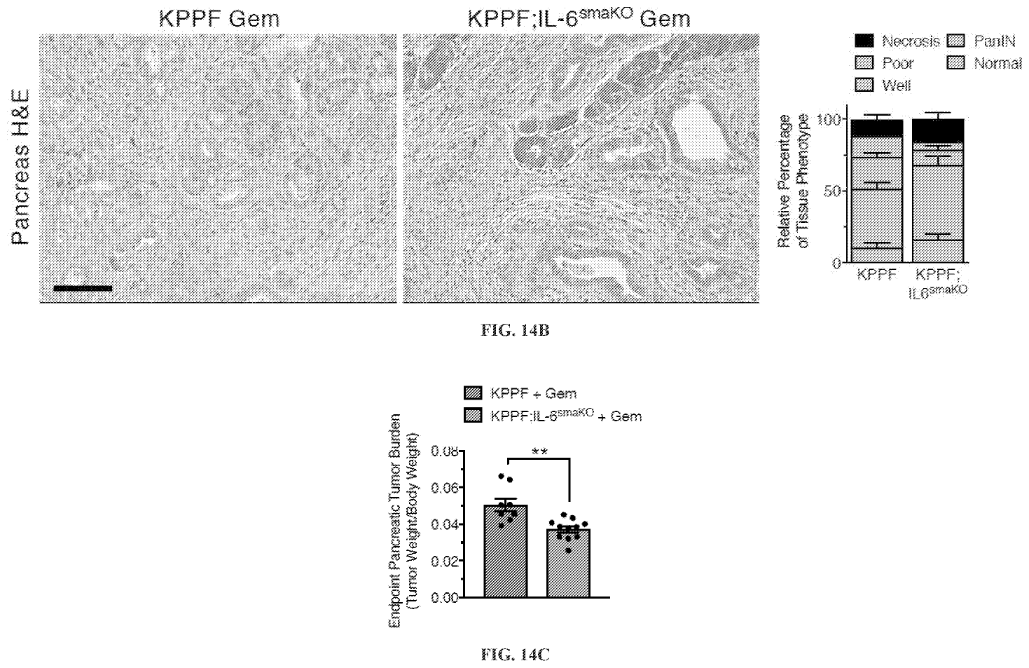

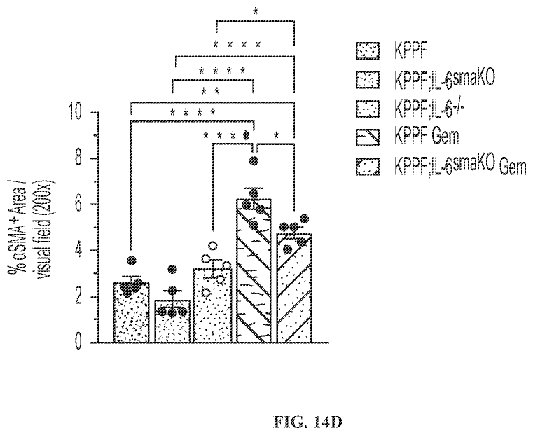

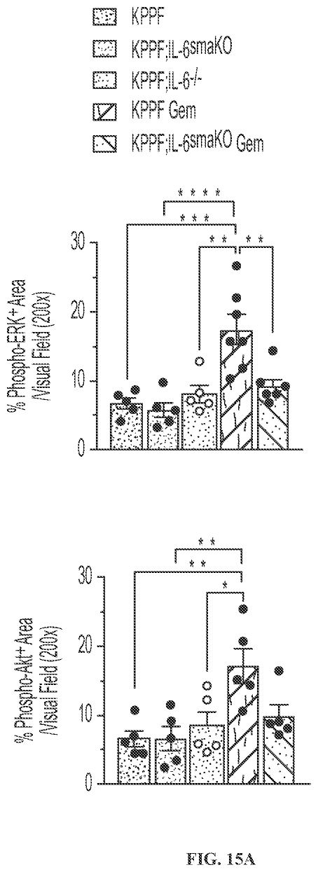

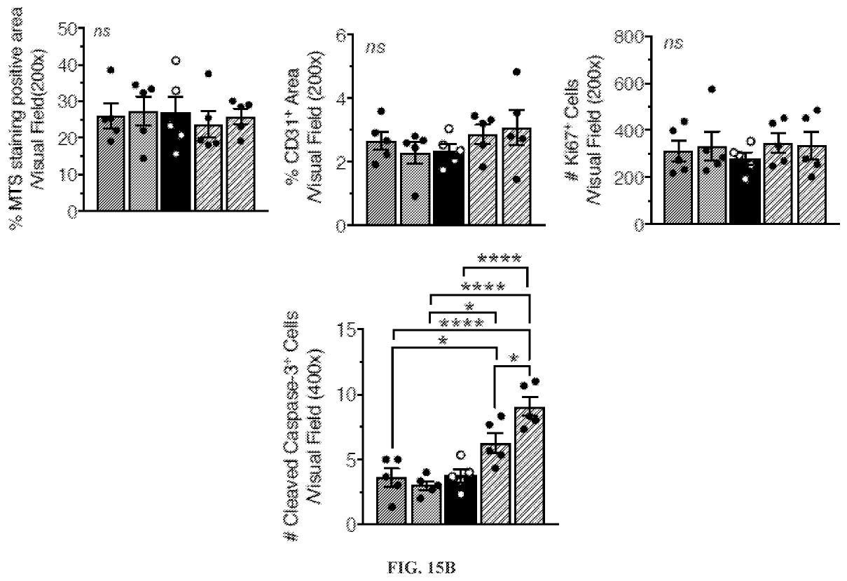

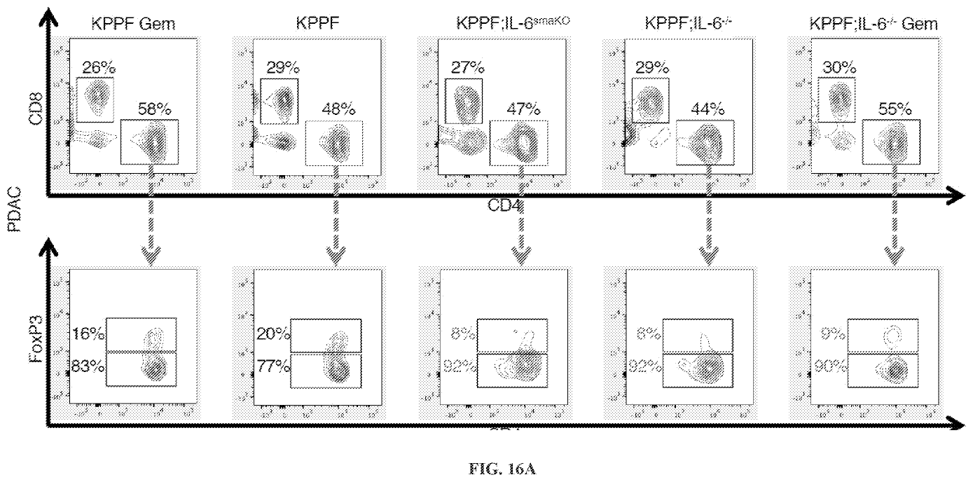

Systemic loss of IL-6 or αSMA⁺ cells-specific loss of IL-6 did not impact disease progression in KPF mice (heterozygous for p53 loss, FIGS. 13A-C). Gemcitabine treatment of mice with PDAC tumors lacking p53 (KPPF) showed no benefit compared to untreated KPPF mice, but mice with reduced (anti-IL-6 neutralizing antibody, αIL-6) or absent (IL-6^{-/-}) IL-6, responded to gemcitabine treatment with increased overall survival (FIGS. 5D, 5F, FIG. 14A). Critically, αSMA⁺ CAFs-specific loss of IL-6)(IL-6^{smaKO} was sufficient to confer the increase in overall survival upon gemcitabine treatment, similar to KPPF mice lacking total IL-6 (IL-6^{-/-}) (FIG. 5F). These results suggest that αSMA⁺ CAFs derived IL-6 confers tumor resistance to gemcitabine. Loss of IL-6 from αSMA⁺ CAFs resulted in improved histopathology and reduced tumor burden in the context of gemcitabine treatment (FIGS. 14B-C). Notably, IL-6 transcript levels and positive cells, as well as αSMA transcript levels and αSMA⁺ CAFs were elevated following gemcitabine treatment (FIG. 5G, FIG. 14D). Cancer cells in tumors of mice treated with gemcitabine showed elevated levels of phosphorylated Stat3, ERK1/2, and Akt, and these changes were significantly attenuated with loss of IL-6 from αSMA⁺ CAFs (FIG. 5H, FIG. 15A). Gemcitabine treatment in mice with αSMA⁺ CAFs-specific loss of IL-6 did not significantly impact tumor collagen deposition, vasculature, or cancer cell proliferation compared to controls; however, cleaved caspase-3, indicative of apoptosis, was elevated in mice treated with gemcitabine, and further increased in mice treated with gemcitabine concurrent with αSMA⁺ CAFs-specific loss of IL-6 (FIG. 15B).

### Example 7—Opportunistic Response to Immune Checkpoint Blockade when Coupled with IL-6 Suppression and Gemcitabine Treatment

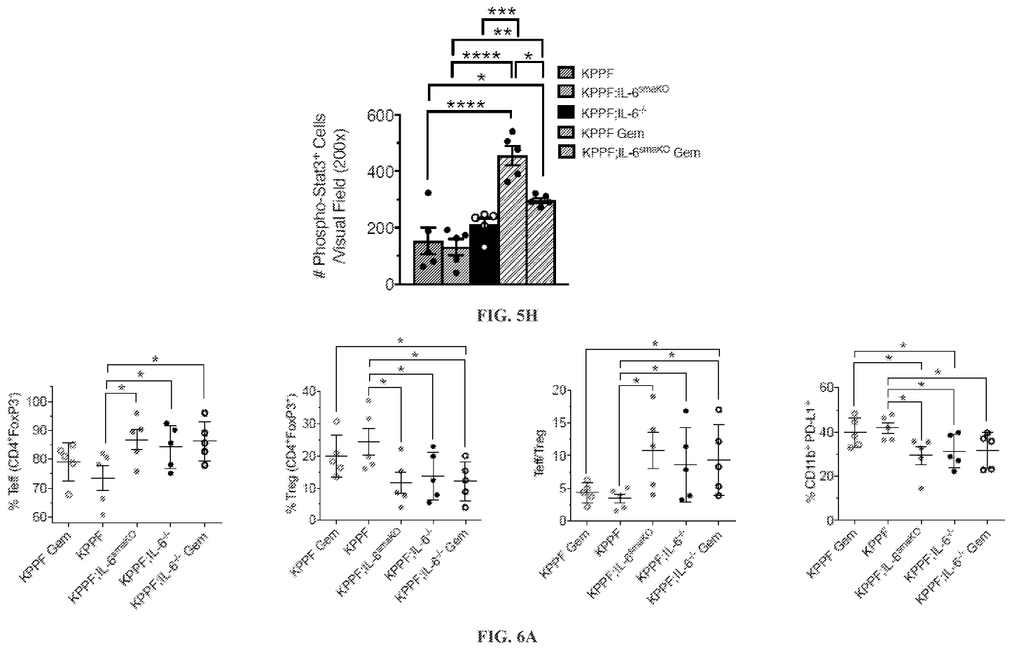

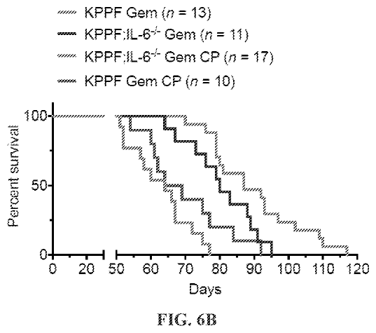

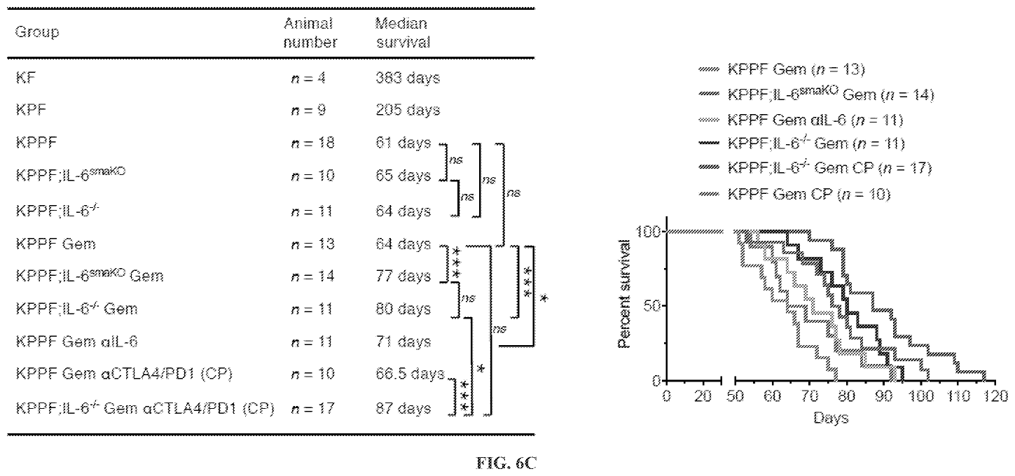

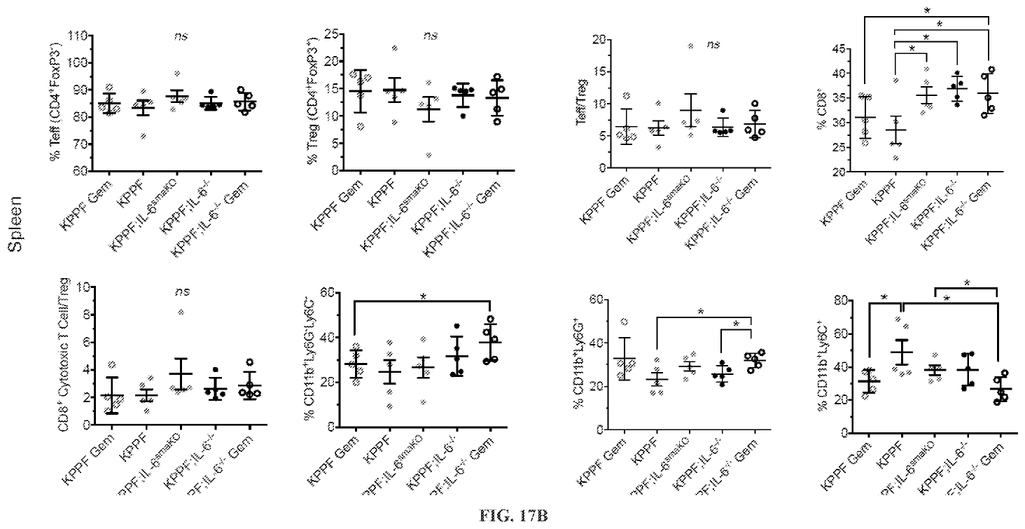

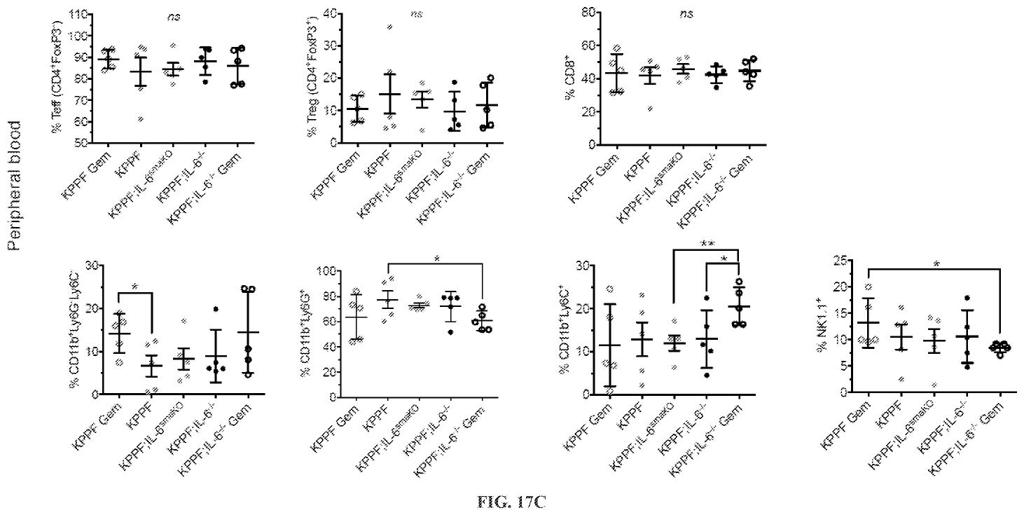

The result gathered in this report suggest a self-preserving/pro-survival program launched by αSMA⁺ CAFs (enhancing IL-6) when tumors encounter treatment with chemotherapeutics such as gemcitabine. In such a scenario, cancer cells could use the IL-6 produced by αSMA⁺ CAFs to induce pro-survival signals and also manipulate the immune system to induce immunosuppression. To address this hypothesis, the immune composition of tumor, spleen, and peripheral blood of KPPF, KPPF;IL-6^{-/-}, and KPPF;IL-6^{smaKO} mice, with and without gemcitabine therapy, was evaluated (FIGS. 16A-C). Intra-tumoral immune cell frequencies were dominantly impacted by loss of IL-6 compared to spleen and peripheral blood immune frequencies (FIG. 6A, FIGS. 17A-C). Gemcitabine treatment minimally impacted intra-tumoral T cell frequencies with IL-6. The number of regulatory T cells (Treg) and effector T cells (Teff) significantly changed, elevating the Teff/Treg ratio in both KPPF;IL-6^{-/-} and KPPF;IL-6^{smaKO} mice (FIG. 6A). Further, the frequency of CD11b⁺PD-L1⁺ cells were significantly reduced in KPPF;IL-6^{-/-} and KPPF;IL-6^{smaKO} mice compared to control KPPF mice (FIG. 6A). Importantly, while the loss of αSMA⁺ CAFs-derived IL-6 was sufficient to increase the Teff/Treg ratio and suppress the frequencies of immunosuppressive CD11b⁺PD-L1⁺ cells, such tumor immunity profile did not translate into improved survival of mice (FIGS. 6B-C). Nevertheless, overall increase in survival was observed in KPPF;IL-6^{-/-} and KPPF;IL-6^{smaKO} mice treated with gemcitabine when compared to the control KPPF mice treated with gemcitabine. Such benefit could be in part due improved tumor immunity due to the loss of IL-6. Next, the therapeutic benefit of dual inhibition of immune checkpoint blockade (anti-CTLA4 and anti-PD-1; αCP) was tested in the context of IL-6 loss. The results indicated that the combination of IL-6 loss with gemcitabine and anti-CTLA4 and anti-PD-1 therapy yield significant benefit (FIGS. 6B-C). This combination therapy concurrent with IL-6 suppression emerged as the most effective combination with significant impact in increasing the overall survival of mice with PDAC (FIGS. 6B-C).

Tumor IL-6 was evaluated in the pancreatic cancer transcriptome analysis reported in the TCGA database. Patients were stratified into two groups based on the median IL-6 expression level; IL-6 high (n=90 cases) and IL-6 low (n=89 cases). Interestingly, tumors in the IL-6 high group also showed high FOXP3 mRNA levels, whereas GAPDH and ACTA2 (αSMA) transcripts were not significantly altered (FIG. 6D). These data support that notion that transcriptional upregulation of IL-6 in human PDAC tumors correlates with potentially increased Tregs (FIG. 6A).

TABLE 1
Genetically Engineered Mice (GEM) nomenclature

| Nomen- | | |
|---|---|---|
| clature | Definition | Reference |
| PKT | Ptf1a^{cre/+;} LSL-Kras^{G12D/+}; | (LeBleu et al., 2013; |
| | Tgfbr2^{loxP/loxP} | Ozdemir et al., 2015) |
| PKTY; | Ptf1a^{cre/+;} LSL-Kras^{G12D/+}; | (LeBleu et al., 2013; |
| αSMA- | Tgfbr2^{loxP/loxP}; Rosa26-LSL-YFP; | Ozdemir et al., 2015) |
| RFP | αSMA-RFP | |
| PKT; | Ptf1a^{cre/+;} LSL-Kras^{G12D/+}; | (LeBleu et al., 2013; |
| αSMA-TK | Tgfbr2^{loxP/loxP}; αSMA-TK (enables | Ozdemir et al., 2015) |
| | depletion of proliferating αSMA⁺ | |
| | cells with GCV) | |
| PKT; FAP- | Ptf1a^{cre/+;} LSL-Kras^{G12D/+}; | (Ozdemir et al., |
| TK | Tgfbr2^{loxP/loxP}; FAP-TK (enables | 2015), Kalluri |
| | depletion of proliferating FAP⁺ | laboratory |
| | cells with GCV) | |
| FAP-TK | | Kalluri laboratory |
| PKP | Ptf1a^{cre/+;} LSL-Kras^{G12D/+}; Trp53^{R172H/+} | |
| PKP; | Ptf1a^{cre/+;} LSL-Kras^{G12D/+}; | (Ozdemir |
| αSMA-TK | Trp53^{R172H/+}; αSMA-TK (enables | et al., 2015) |
| | depletion of proliferating αSMA⁺ | |
| | cells with GCV) | |
| KF | FSF-Kras^{G12D/+}; Pdx1^{Flp/+} | (Schonhuber |
| | | et al., 2014) |
| KPF | FSF-Kras^{G12D/+}; Trp53^{frt/+}; Pdx1^{Flp/+} | (Schonhuber |
| | | et al., 2014) |
| KPPF | FSF-Kras^{G12D/+}; Trp53^{frt/frt}; Pdx1^{Flp/+} | (Schonhuber |
| | | et al., 2014) |
| PPF | FSF-Kras^{G12D/+}; Trp53^{frt/frt} | (Schonhuber |
| KPPC | LSL-Kras^{G12D/+}; Trp53^{loxP/loxP}; | et al., 2014) |
| | Pdx1^{cre/+} | |
| KPPF; | FSF-Kras^{G12D/+}; Trp53^{frt/frt}; Pdx1^{Flp/+}; | (Chen et al., 2018) |
| αSMA- | αSMA-Cre; Rosa26-CAG-loxP-frt- | |
| Cre; | Stop-frt-FireflyLuc-EGFP-loxP- | |
| R26^{Dual} | RenillaLuc-tdTomato | |
| KPPF; | FSF-Kras^{G12D/+}; Trp53^{frt/frt}; Pdx1^{Flp/+}; | |
| αSMA- | αSMA-Cre; Rosa26-CAG- | |
| Cre; | Brainbow 2.1 | |
| R26^{Confetti} | | |
| KF; | FSF-Kras^{G12D/+}; Pdx1^{Flp/+}; αSMA- | (Quintana et al., |
| IL-6^{smaKO} | Cre; IL-6^{loxP/loxP} | 2013) (Schonhuber |
| | | et al., 2014) |
| KF; IL-6^{-/-} | FSF-Kras^{G12D/+}; Pdx1^{Flp/+}; IL-6^{-/-} | |
| KPF; | FSF-Kras^{G12D/+}; Trp53^{frt/+}; Pdx1^{Flp/+}; | |
| IL-6^{smaKO} | αSMA-Cre; IL-6^{loxP/loxP} | |
| KPF; | FSF-Kras^{G12D/+}; Trp53^{frt/+}; Pdx1^{Flp/+}; | |
| IL-6^{-/-} | IL-6^{-/-} | |
| KPPF; | FSF-Kras^{G12D/+}; Trp53^{frt/frt}; Pdx1^{Flp/+}; | |
| IL-6^{smaKO} | αSMA-Cre; IL-6^{loxP/loxP} | |
| KPPF; | FSF-Kras^{G12D/+}; Trp53^{frt/frt}; Pdx1^{Flp/+}; | |
| IL-6^{-/-} | IL-6^{-/-} | |

TABLE 2
Antibodies

| Antibody/Application | Source (catalog, clone) | Dilution |
|---|---|---|
| αSMA/IF (frozen) | DAKO, M0851 | 1:200 |
| FAP/IF (frozen) | Abcam, ab53066 | 1:200 |
| αSMA/IHC | DAKO, M0851 | 1:200 |
| CD31/IHC | Abcam, ab28364 | 1:300 |
| CK19/IHC | Abcam, ab52625 | 1:100 |
| Cleaved caspase-3/IHC | Cell Signaling, 9661 | 1:200 |
| Ki67/IHC | Thermo Scientific, RM-9106 | 1:400 |
| phospho-Akt/IHC | Abcam, Ser473. ab81283 | 1:400 |
| phospho-ERK1/2/IHC | Cell Signaling, Thr202/ | 1:400 |
| | Tyr204. 4695 | |
| phospho-Stat3/IHC | Cell Signaling, Tyr705. 9145 | 1:200 |
| FAP/IHC | Abcam. ab28244 | 1:50 |
| FAP/IHC (human) | R&D, AF3715 | 1:20 |
| αSMA/TSA | DAKO, M0851 | 1:2000 |
| Fsp1/TSA | DAKO, A5114 | 1:6000 |
| CK8/TSA | DSHB, Troma-I | 1:50 |
| Vimentin/TSA | Cell Signaling, CS5741 | 1:1000 |
| PDGFRα/TSA | R&D, AF1062 | 1:200 |
| FAP/FC | Abcam, ab28244 | 1:100 |
| Rabbit IgG, | Abcam, 171870 | 1:100 |
| polyclonal/FC | | |
| Zenon AF647 IgG | ThermoFisher Scientific, Z25308 | 1:100 |
| Labeling Kit/FC | | |
| Fixable Viability Dye | eBioscience, 65-0865-14 | 1:1000 |
| eFluor 780/FC | | |
| CD45.2 Pacific Blue/FC | BioLegend, 103126 (30-F11) | 1:100 |
| CD3 PE-Cy7/FC | eBioscience, 25-0031-82- | 1:200 |
| | (145-2c11) | |
| CD3 Alexa Fluor 700/FC | eBioscience, 56-0032-82 (17A2) | 1:50 |
| FoxP3 Alexa Fluor | eBioscience, 56-5773-82 | 1:50 |
| 700/FC | (FJK-16s) | |
| CD11c eFluor 615/FC | eBioscience, 42-0114-82 (N418) | 1:50 |
| NK1.1 PE/FC | eBioscience, 12-5941-83 (PK136) | 1:200 |
| CD4 Qdot 605/FC | BioLegend, 100548 (RM4-5) | 1:200 |
| CD8 BV650/FC | BioLegend, 100742 (53-6.7) | 1:200 |
| CD11b BV570/FC | BioLegend, BD, 562950 (M1/70) | 1:200 |
| CD19 Qdot655/FC | BioLegend, 115541 (6D5) | 1:100 |
| Ly6C APC/FC | BD Biosciences, 560595 (AL-21) | 1:200 |
| Ly6G PE-Cy7/FC | BD Biosciences, 560601 (1A8) | 1:200 |
| Ki-67 PE/FC | BD Biosciences, 558616 (B56) | 1:100 |

IF: immunofluorescence on frozen sections or FFPE,
IHC: immunohistochemistry on FFPE (FFPE: formalin fixed paraffin embedded)
FC: Flow cytometry
TSA: Tyramide signal amplification (multispectral imaging)
DSHB: Development Studies Hybridoma Bank, University of Iowa
* * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Carstens et al., “Spatial computation of intratumoral T cells correlates with survival of patients with pancreatic cancer,” Nat. Commun., 8:15095 (2017).
Cerami et al., “The cBio cancer genomics portal: an open platform for exploring multidimensional cancer genomics data,” Cancer Discov., 2:401-404 (2012).
Chen et al., “Dual reporter genetic mouse models of pancreatic cancer identify an epithelial-to-mesenchymal transition-independent metastasis program,” EMBO Mol. Med., 10:e9085 (2018).
Feig et al., “Targeting CXCL12 from FAP-expressing carcinoma-associated fibroblasts synergizes with anti-PD-L1 immunotherapy in pancreatic cancer,” Proc. Natl. Acad. Sci. U.S.A., 110:20212-20217 (2013).
Hingorani et al., “Trp53R172H and KrasG12D cooperate to promote chromosomal instability and widely metastatic pancreatic ductal adenocarcinoma in mice,” Cancer Cell, 7:469-483 (2005).
Ijichi et al., “Aggressive pancreatic ductal adenocarcinoma in mice caused by pancreas-specific blockade of transforming growth factor-beta signaling in cooperation with active Kras expression,” Genes Dev., 20:3147-3160 (2006).
Kalluri, “The biology and function of fibroblasts in cancer,” Nat. Rev. Cancer, 16:582-598 (2016).
Kamerkar et al., “Exosomes facilitate therapeutic targeting of oncogenic KRAS in pancreatic cancer,” Nature, 546:498-503 (2017).
Kraman et al., “Suppression of antitumor immunity by stromal cells expressing fibroblast activation protein-alpha,” Science, 330:827-830 (2010).
LeBleu et al., “Origin and function of myofibroblasts in kidney fibrosis,” Nat. Med., 19:1047-1053 (2013).
LeBleu & Kalluri, “A peek into cancer-associated fibroblasts: origins, functions and translational impact,” Dis. Model Mech., 11:dmm029447 (2018).
Lee et al., “Generation of primary tumors with Flp recombinase in FRT-flanked p53 mice,” Dis. Model Mech., 5:397-402 (2012).
Lesina et al., “Interleukin-6 in inflammatory and malignant diseases of the pancreas,” Semin. Immunol., 26:80-87 (2014).
Lo et al., “Fibroblast activation protein augments progression and metastasis of pancreatic ductal adenocarcinoma,” JCI Insight, 2:92232 (2017).
Meyerholz & Beck, “Principles and approaches for reproducible scoring of tissue stains in research,” Lab. Invest., 98:844-855 (2018).
Nagathihalli et al., “Pancreatic stellate cell secreted IL-6 stimulates STATS dependent invasiveness of pancreatic intraepithelial neoplasia and cancer cells,” Oncotarget, 7:65982-65992 (2016).
Neesse et al., “Stromal biology and therapy in pancreatic cancer: a changing paradigm,” Gut, 64:1476-1484 (2015).
Ohlund et al., “Fibroblast heterogeneity in the cancer wound,” J. Exp. Med., 211:1503-1523 (2014).
Ohlund et al., “Distinct populations of inflammatory fibroblasts and myofibroblasts in pancreatic cancer,” J. Exp. Med., 214:579-596 (2017).
Ozdemir et al., “Depletion of carcinoma-associated fibroblasts and fibrosis induces immunosuppression and accelerates pancreas cancer with reduced survival,” Cancer Cell, 25:719-734 (2014).
Quintana et al., “Astrocyte-specific deficiency of interleukin-6 and its receptor reveal specific roles in survival, body weight and behavior,” Brain, Behavior, and Immunity, 27:162-173 (2013).
Rhim et al., “Stromal elements act to restrain, rather than support, pancreatic ductal adenocarcinoma,” Cancer Cell, 25:735-747 (2014).
Roberts et al., “Depletion of stromal cells expressing fibroblast activation protein-alpha from skeletal muscle and bone marrow results in cachexia and anemia,” J. Exp. Med., 210:1137-1151 (2013).
Schonhuber et al., “A next-generation dual-recombinase system for time- and host-specific targeting of pancreatic cancer,” Nat. Med., 20:1340-1347 (2014).
Smyth, “limma: Linear Models for Microarray Data,” In: Gentleman R., Carey V. J., Huber W., Irizarry R. A., Dudoit S. (eds), Bioinformatics and Computational Biology Solutions Using R and Bioconductor, Statistics for Biology and Health, Springer, New York, N.Y. (2005).
Subramanian et al., “Gene set enrichment analysis: a knowledge-based approach for interpreting genome-wide expression profiles,” Proc. Natl. Acad. Sci. U.S.A., 102:15545-15550 (2005).
Tran et al., “Immune targeting of fibroblast activation protein triggers recognition of multipotent bone marrow stromal cells and cachexia,” J. Exp. Med., 210:1125-1135 (2013).
Wormann et al., “Loss of P53 Function Activates JAK2-STAT3 Signaling to Promote Pancreatic Tumor Growth, Stroma Modification, and Gemcitabine Resistance in Mice and Is Associated With Patient Survival,” Gastroenterology, 151:180-193.e12 (2016).
Yang et al., “FAP Promotes Immunosuppression by Cancer-Associated Fibroblasts in the Tumor Microenvironment via STAT3-CCL2 Signaling,” Cancer Res., 76:4124-4135 (2016).
Zheng et al., “Epithelial-to-mesenchymal transition is dispensable for metastasis but induces chemoresistance in pancreatic cancer,” Nature, 527:525-530 (2015).

1. A method of treating a subject having cancer, the method comprising administering an anti-tumor effective amount of a composition that comprises an agent that suppresses IL-6 signaling, gemcitabine, and an immune checkpoint blockade therapy.

2. (canceled)

3. The method of claim 1, wherein the subject has previously failed to respond to immune checkpoint blockade therapy.

4. The method of claim 1, further comprising administering an effective amount of an antibody or an antibody fragment or a chimeric antigen receptor that binds to a protein that is expressed by TP-CAFs and is not expressed by TS-CAFs.

5. The method of claim 1, wherein the cancer is a pancreatic cancer.

6. (canceled)

7. (canceled)

8. The method of claim 1, further comprising administering at least a second anti-cancer therapy.

9. The method of claim 8, wherein the second anti-cancer therapy is a chemotherapy, immunotherapy, radiotherapy, gene therapy, surgery, hormonal therapy, anti-angiogenic therapy or cytokine therapy.

10. A composition comprising an antibody or an antibody fragment or a chimeric antigen receptor that binds to a protein that is expressed by TP-CAFs and is not expressed by TS-CAFs.

11. The composition of claim 10, wherein the composition comprises an antibody fragment, wherein the antibody fragment is a recombinant scFv (single chain fragment variable) antibody, Fab fragment, F(ab′)₂ fragment, or Fv fragment.

12. The composition of claim 10, wherein the composition comprises an antibody, wherein the antibody is a bispecific antibody.

13. (canceled)

14. The composition of claim 12, wherein the bispecific antibody binds to both (1) a protein that is expressed by TP-CAFs and is not expressed by TS-CAFs and (2) CD3.

15. The composition of claim 10, wherein the antibody or antibody fragment is conjugated to a cytotoxic agent or a diagnostic agent.

16. (canceled)

17. The composition of claim 10, wherein the composition comprises a hybridoma or engineered cell encoding the antibody or antibody fragment or chimeric antigen receptor.

18. (canceled)

19. A method of treating a patient in need thereof, the method comprising administering an effective amount of the composition of claim 10.

20. (canceled)

21. The method of claim 19, wherein the patient has cancer.

22. The method of claim 21, wherein said patient has been determined to comprise FAP⁺ CAFs.

23. The method of claim 21, wherein the cancer is pancreatic cancer.

24. (canceled)

25. (canceled)

26. The method of claim 19, further comprising administering at least a second anti-cancer therapy.

27. The method of claim 26, wherein the second anti-cancer therapy is a chemotherapy, immunotherapy, radiotherapy, gene therapy, surgery, hormonal therapy, anti-angiogenic therapy or cytokine therapy.

28. (canceled)

29. The composition of claim 10, wherein the composition comprises a chimeric antigen receptor comprising an antigen binding domain that binds to a protein that is expressed by TP-CAFs and is not expressed by TS-CAFs, wherein the antigen binding domain comprises HCDR sequences from a first antibody that binds to a protein that is expressed by TP-CAFs and is not expressed by TS-CAFs and LCDR sequences from a second antibody that binds to a protein that is expressed by TP-CAFs and is not expressed by TS-CAFs.

30. The composition of claim 10, wherein the antigen binding domain comprises HCDR sequences and LCDR sequence from an antibody that binds to a protein that is expressed by TP-CAFs and is not expressed by TS-CAFs.

31.-35. (canceled)

36. The composition of claim 10, wherein the composition comprises an isolated immune effector cell comprising the antibody or antibody fragment or chimeric antigen receptor.

37.-47. (canceled)

48. A method of treating a subject comprising administering an anti-tumor effective amount of the composition of claim 36, wherein the immune effector cell is a chimeric antigen receptor (CAR) T cell or a CAR NK cell.

49. The method of claim 48, wherein the immune effector cell is allogeneic.

50. The method of claim 48, wherein the immune effector cell is autologous.

51. The method of claim 48, wherein the immune effector cell is HLA matched to the subject.

52. The method of claim 48, wherein the subject has cancer.

53. The method of claim 52, wherein the cancer is pancreatic cancer.

54. A method of cancer tissue analysis, the method comprising contacting a cancer tissue obtained from a subject with the composition of claim 10.


SEQUENCE LISTING (8 sequences)

SEQ ID NO: 1 - DNA, 21 bp, Artificial Sequence
  Synthetic polynucleotide
aggtcggtgtgaacggatttg

SEQ ID NO: 2 - DNA, 23 bp, Artificial Sequence
  Synthetic polynucleotide
tgtagaccatgtagttgaggtca

SEQ ID NO: 3 - DNA, 27 bp, Artificial Sequence
  Synthetic polynucleotide
gcttaattacacatgttctctgggaaa

SEQ ID NO: 4 - DNA, 24 bp, Artificial Sequence
  Synthetic polynucleotide
caagtgcatcatcgttgttcatac

SEQ ID NO: 5 - DNA, 20 bp, Artificial Sequence
  Synthetic polynucleotide
gggctgcttccaaacctttg

SEQ ID NO: 6 - DNA, 22 bp, Artificial Sequence
  Synthetic polynucleotide
tgatactgcctgcctgaagctc

SEQ ID NO: 7 - DNA, 21 bp, Artificial Sequence
  Synthetic polynucleotide
gtcccagacatcagggagtaa

SEQ ID NO: 8 - DNA, 21 bp, Artificial Sequence
  Synthetic polynucleotide
tcggatacttcagcgtcagga